SEQUENCE LISTING (50 sequences)

SEQ ID NO: 1 - protein, 8 aa, Artificial
  Synthetic KB-AT-001  CDR1 SEQ ID NO 1
GRTFRNYV

SEQ ID NO: 2 - protein, 8 aa, Artificial
  Synthetic KB-AT-001 CDR2 SEQ ID NO 2
INRSGAIT

SEQ ID NO: 3 - protein, 16 aa, Artificial
  Synthetic KB-AT-001 CDR3 SEQ ID NO 3
AAGETTWSIRRDDYDY

SEQ ID NO: 4 - protein, 123 aa, Artificial
  Synthetic KB-AT-001 SEQ ID NO 4
QVQLQQSGGDLAQRGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 5 - protein, 9 aa, Artificial
  Synthetic KB-AT-002 CDR1 SEQ ID NO 5
SGRTFNNNG

SEQ ID NO: 6 - protein, 8 aa, Artificial
  Synthetic KB-AT-002 CDR2 SEQ ID NO 6
ISWSGGST

SEQ ID NO: 7 - protein, 17 aa, Artificial
  Synthetic KB-AT-002 CDR 3 SEQ ID NO 7
AARTRYNSGLFSRNYDY

SEQ ID NO: 8 - protein, 124 aa, Artificial
  Synthetic KB-AT-002 SEQ ID NO 8
QVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 9 - protein, 9 aa, Artificial
  Synthetic KB-AT-003 CDR1 SEQ ID NO 9
ALTFSSRAW

SEQ ID NO: 10 - protein, 8 aa, Artificial
  Synthetic KB-AT-003 CDR2 SEQ ID NO 10
ITGGGTTN

SEQ ID NO: 11 - protein, 8 aa, Artificial
  Synthetic KB-AT-003 CDR3 SEQ ID NO 11
NGYRYTYA

SEQ ID NO: 12 - protein, 114 aa, Artificial
  Synthetic KB-AT-003 SEQ ID NO 12
QVQLVQSGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSS

SEQ ID NO: 13 - protein, 7 aa, Artificial
  Synthetic KB-AT-004  CDR1 SEQ ID NO 13
AMTFSIR

SEQ ID NO: 14 - protein, 7 aa, Artificial
  Synthetic KB-AT-004  CDR2 SEQ ID NO 14
IGTGDIT

SEQ ID NO: 15 - protein, 8 aa, Artificial
  Synthetic KB-AT-004 CDR3 SEQ ID NO 15
NGYRSTYA

SEQ ID NO: 16 - protein, 113 aa, Artificial
  Synthetic KB-AT-004 SEQ ID NO 16
VQLQQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 17 - protein, 9 aa, Artificial
  Synthetic KB-AT-005  CDR1 SEQ ID NO 17
GRDFNDAAL

SEQ ID NO: 18 - protein, 7 aa, Artificial
  Synthetic KB-AT-005 CDR2 SEQ ID NO 18
ITSGGVR

SEQ ID NO: 19 - protein, 16 aa, Artificial
  Synthetic KB-AT-005  CDR3 SEQ ID NO 19
KADSFKGDYDTSWYLY

SEQ ID NO: 20 - protein, 122 aa, Artificial
  Synthetic KB-AT-005 SEQ ID NO 20
EVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 21 - protein, 8 aa, Artificial
  Synthetic KB-AT-006  CDR1 SEQ ID NO 21
GRTFSNNG

SEQ ID NO: 22 - protein, 8 aa, Artificial
  Synthetic KB-AT-006 CDR2 SEQ ID NO 22
ISWSSGST

SEQ ID NO: 23 - protein, 17 aa, Artificial
  Synthetic KB-AT-006  CDR3 SEQ ID NO 23
AARTRYNSGYFTRNYDY

SEQ ID NO: 24 - protein, 124 aa, Artificial
  Synthetic KB-AT-006 SEQ ID NO 24
QVQLQQSGGGLVQAGGSLRLSCAASGRTFSNNGMGWFRQAPGKEREFVAAISWSSGSTYYADSVKGRYTISRDNAKNTVYLQMNSLKPEDTAVYYCAARTRYNSGYFTRNYDYWGQGTQVTVSS

SEQ ID NO: 25 - protein, 8 aa, Artificial
  Synthetic KB-AT-007 CDR1 SEQ ID NO 25
GRTFRNYV

SEQ ID NO: 26 - protein, 8 aa, Artificial
  Synthetic KB-AT-007 CDR2 SEQ ID NO 26
INRSGAIT

SEQ ID NO: 27 - protein, 16 aa, Artificial
  Synthetic KB-AT-007 CDR3 SEQ ID NO 27
AAGETTWSIRRDDYDY

SEQ ID NO: 28 - protein, 123 aa, Artificial
  Synthetic KB-AT-007 SEQ ID NO 28
QVQLQQSGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 29 - protein, 254 aa, Artificial
  Synthetic SEQUENCE KB-AT-002/003 SEQ ID NO 29
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSS

SEQ ID NO: 30 - protein, 263 aa, Artificial
  Synthetic SEQUENCE KB-AT-001/002 SEQ ID NO 30
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 31 - protein, 256 aa, Artificial
  Synthetic SEQUENCE KB-AT-001/003 SEQ ID NO 31
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 32 - protein, 261 aa, Artificial
  Synthetic SEQUENCE KB-AT-001/005 SEQ ID NO 32
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSEVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 33 - protein, 402 aa, Artificial
  Synthetic SEQUENCE KB-AT-112 SEQ ID NO 33
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 34 - protein, 395 aa, Artificial
  Synthetic SEQUENCE  KB-AT-113 SEQ ID NO 34
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 35 - protein, 400 aa, Artificial
  Synthetic SEQUENCE  KB-AT-115 SEQ ID NO 35
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSEVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 36 - protein, 535 aa, Artificial
  Synthetic KB-AT-1123 SEQ ID NO 36
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 37 - protein, 486 aa, Artificial
  Synthetic SEQUENCE  VWF-A1/KB-AT-002/003 SEQ ID      NO 37
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSSGRGGGSGGGSGGGSDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLAYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTV

SEQ ID NO: 38 - protein, 217 aa, Artificial
  Synthetic KB-AT-002/C4BP SEQ ID NO 38
MVPARFAGVLLALALILPGTLCQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSSGETPEGCEQVLTGKRLMQCLPNPEDVKMALEVYKLSLEIEQLELQRDSARQSTLDKELEDQVDPRLIDGK

SEQ ID NO: 39 - protein, 207 aa, Artificial
  Synthetic KB-AT-003/-C4BP SEQ ID NO 39
MVPARFAGVLLALALILPGTLCQVQLQQSGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSSSGETPEGCEQVLTGKRLMQCLPNPEDVKMALEVYKLSLEIEQLELQRDSARQSTLDKELEDQVDPRLIDGK

SEQ ID NO: 40 - protein, 734 aa, Artificial
  Synthetic SEQUENCE  mFVII-AT-0203 SEQ ID NO 40
MVPQAHGLLLLCFLLQLQGPLGTAVFITQEEAHGVLHRQRRANSLLEELWPGSLERECNEEQCSFEEAREIFKSPERTKQFWIVYSDGDQCASNPCQNGGTCQDHLKSYVCFCLLDFEGRNCEKSKNEQLICANENGDCDQYCRDHVGTKRTCSCHEDYTLQPDEVSCKPKVEYPCGRIPVVEKRNSSSRQGRRKRRKRLVGGNVCPKGECPWQAVLKINGLLLCGAVLLDARWIVTAAHCFDNIRYWGNITVVMGEHDFSEKDGDEQVRRVTQVIMPDKYIRGKINHDIALLRLHRPVTFTDYVVPLCLPEKSFSENTLARIRFSRVSGWGQLLDRGATALELMSIEVPRLMTQDCLEHAKHSSNTPKITENMFCAGYMDGTKDACKGDSGGPHATHYHGTWYLTGVVSWGEGCAAIGHIGVYTRVSQYIDWLVRHMDSKLQVGVFRLPLLLTPRGVRLGGGSQVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSSGGGSEDQVDPRLIDGK

SEQ ID NO: 41 - protein, 1708 aa, Artificial
  Synthetic SEQUENCE  FVIII-AT-0203 SEQ ID NO 41
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSGGGSQVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSSGGGSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMEWMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 42 - protein, 391 aa, Artificial
  Synthetic SEQUENCE  KB-AT-114  SEQ ID NO 42
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 43 - protein, 381 aa, Artificial
  Synthetic SEQUENCE KB-AT-644 SEQ ID NO 43
QVQLQSGGGLVQAGGSLRLSCAASGRTFSNNGMGWFRQAPGKEREFVAAISWSSGSTYYADSVKGRYTISRDNAKNTVYLQMNSLKPEDTAVYYCAARTRYNSGYFTRNYDYWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 44 - protein, 382 aa, Artificial
  Synthetic SEQUENCE KB-AT-244 SEQ ID NO 44
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 45 - protein, 375 aa, Artificial
  Synthetic SEQUENCE KB-AT-443  SEQ ID NO 45
QVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 46 - protein, 255 aa, Artificial
  Synthetic  SEQUENCE  KB-AT-002004 SEQ ID NO 46
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVTVSS

SEQ ID NO: 47 - protein, 242 aa, Artificial
  Synthetic SEQUENCE KB-AT-004004 SEQ ID NO 47
QVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSSGGGSGGGSGGGSGGGSQVQLQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 48 - protein, 261 aa, Artificial
  Synthetic SEQUENCE  KB-AT-002006  SEQ ID NO 48
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQSGGGFVQAGGSLRLSCAASGRTFSNNGMGWFRQAPGKEREFVAAISWSSGSTYYADSVKGRYTISSDNAKNTVYLQMNSLKPEDTAVYYCAARTRYNRGYFTRNYDYWGQGTQVTV

SEQ ID NO: 49 - protein, 262 aa, Artificial
  Synthetic SEQUENCE  KB-AT-001001 SEQ ID NO 49
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 50 - protein, 535 aa, Artificial
  Synthetic SEQUENCE KB-AT-6623 SEQ ID NO 50
QVQLQSGGGLVQAGGSLRLSCAASGRTFSNNGMGWFRQAPGKEREFVAAISWSSGSTYYADSVKGRYTISRDNAKNTVYLQMNSLKPEDTAVYYCAARTRYNSGYFTRNYDYWGQGTQVTVSSGGGSGGGSGGGSGGGSQVQLQSGGGFVQAGGSLRLSCAASGRTFSNNGMGWFRQAPGKEREFVAAISWSSGSTYYADSVKGRYTISSDNAKNTVYLQMNSLKPEDTAVYYCAARTRYNRGYFTRNYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

(19) United States
(12) Patent Application Publication
(10) Pub. No.: US 2019/0100602 A1
(43) Pub. Date: Apr. 4, 2019

(54) ANTI-ANTITHROMBIN SINGLE-DOMAIN ANTIBODIES AND POLYPEPTIDES COMPRISING THEREOF

(71) Applicants: INSERM (INSTITUT NATIONAL DE LA SANTÉ ET DE LA RECHERCHE MÉDICALE, Paris (FR); UNIVERSITÉ PARIS-SUD, Orsay (FR)

(72) Inventors: Peter LENTING, Kremlin-Bicêtre (FR); Cécile DENIS, Kremlin-Bicêtre (FR); Olivier CHRISTOPHE, Kremlin-Bicêtre (FR); Gabriel AYME, Kremlin-Bicêtre (FR)

(21) Appl. No.: 16/086,146

(22) PCT Filed: Mar. 17, 2017
(86) PCT No.: PCT/EP2017/056424

§ 371 Date: Sep. 18, 2018

(30) Foreign Application Priority Data
Mar. 18, 2016 (EP) 16305301.0

(51) Int. Cl. C07K 16/36; C07K 16/38; A61P 7/04; A61K 38/17; C07K 14/47; C12N 15/62

(52) U.S. Cl. CPC C07K 16/36; C07K 16/38; C12N 15/62; A61K 38/1725; C07K 14/472; A61P 7/04

(57) ABSTRACT

The present invention relates to isolated single-domain antibodies (sdAb) directed against Antithrombin (AT) to prolong the half-life of the proteins. Inventors have generated isolated single domain antibodies (sdAbs) directed against antithrombin. They observed that in amidolytic assays, sdAbs are incapable of blocking the inhibitory antithrombin activity towards thrombin and factor Xa in the presence of heparin. The different combinations of sdAb were able to block the inhibitory antithrombin activity towards thrombin and factor Xa in mice. Thus, the inventors propose to use different combinations of sdAb to block the inhibitory function of antithrombin in order to promote thrombin generation and thus treat haemophilia and other conditions that are associated with bleeding. Accordingly, the invention relates also to a method of preventing or treating bleeding disorders in a subject in need thereof, comprising administering to said subject a therapeutically effective amount of the single domain antibodies or the drug conjugate of the invention.

### FIELD OF THE INVENTION

The invention is in the field of immunotherapy. More particularly, the invention relates to isolated single-domain antibodies (sdAb) directed against Antithrombin (AT) to prolong the half-life of proteins.

The invention relates also to isolated single-domain antibodies (sdAb) directed against Antithrombin (AT) and polypeptides comprising thereof such as blood clotting factors and their uses in therapy such as in the prevention and treatment of hemostatic disorders.

### BACKGROUND OF THE INVENTION

The use of polypeptides such as proteins for therapeutic applications has expanded in recent years mainly due to advanced knowledge of the molecular biological principles underlying many diseases and the availability of improved recombinant expression and delivery systems for human polypeptides. Polypeptide therapeutics are mainly utilized in diseases where a certain natural polypeptide is defective or missing in the patient, in particular because of inherited gene defects. For example, hemophilia is a disease caused by deficiency of a certain plasma protein. Patients having hemophilia suffer from hemorrhagic morbidity caused by the disturbed function of protein components of the blood coagulation cascade. Depending on the affected clotting factor two types of hemophilia can be distinguished. Both have in common the inhibited conversion of soluble fibrinogen to an insoluble fibrin-clot.

In the prior art, the short circulating half-life of polypeptide therapeutics has been addressed by covalent attachment of a polymer to the polypeptide. For example, the attachment of polyethylene glycol (PEG), dextran, or hydroxyethyl starch (HES) has shown some improvement of the half-life of some polypeptides. However, a number of problems have been observed with the attachment of polymers. For example, the attachment of polymers can lead to decreased drug activity. Furthermore, certain reagents used for coupling polymers to a protein are insufficiently reactive and therefore require long reaction times during which protein denaturation and/or inactivation can occur. Also, incomplete or non-uniform attachment leads to a mixed population of compounds having differing properties. WO 2009/135888 discloses a complex comprising a target protein and at least one binding molecule wherein the binding molecule is bound to at least one water soluble polymer.

There is still a need to develop new products that increase the half-life of the therapeutic proteins to increase efficiency or reduce the amount of therapeutic proteins and/or frequency of infusions applied to patient. This would also reduce the costs of the treatment.

### SUMMARY OF THE INVENTION

The invention relates to an isolated single-domain antibodies (sdAb) directed against Antithrombin (AT) to prolong the half-life of proteins. In particular, the present invention is defined by the claims.

### DETAILED DESCRIPTION OF THE INVENTION

Inventors have generated isolated single domain antibodies (sdAbs) directed against antithrombin. They observed that in amidolytic assays, sdAbs are incapable of blocking the inhibitory antithrombin activity towards thrombin and factor Xa in the presence of heparin. Surprisingly, the different combinations of sdAb were able to block the inhibitory antithrombin activity towards thrombin and factor Xa. Thus, the inventors propose to use different combinations of sdAb to block the inhibitory function of antithrombin in order to promote thrombin generation and thus treat haemophilia and other conditions that are associated with bleeding.

In addition to these results on bleeding conditions, inventors have shown that these different combinations could be used to increase the half-life of therapeutic proteins such as the half-life of therapeutic proteins used for the treatment of haemophilia.

In a first aspect, the invention relates to an isolated single domain antibody (sdAb) directed against antithrombin (AT).

By “isolated” it is meant, when referring to a single-domain antibody according to the invention, that the indicated molecule is present in the substantial absence of other biological macromolecules of the same type.

As used herein the term “single-domain antibody” (sdAb) has its general meaning in the art and refers to the single heavy chain variable domain of antibodies of the type that can be found in Camelid mammals which are naturally devoid of light chains. Such single-domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al, Trends Biotechnol, 2003, 21(11):484-490; and WO 06/030220, WO 06/003388. The amino acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementary Determining Region 1” or “CDR1”; as “Complementarity Determining Region 2” or “CDR2” and as “Complementarity Determining Region 3” or “CDR3”, respectively. Accordingly, the single-domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single-domain antibody are numbered according to the general numbering for VH domains given by the International ImMunoGeneTics information system aminoacid numbering (http://imgt.cines.fr/).

Antithrombin (AT) is an anticoagulant factor which prevents the coagulation of blood. It inhibits thrombin, FXa and other serine proteases functioning in the coagulation pathway. It consists of 432 amino acids, is produced by the liver hepatocyte and has a long plasma half-life of two and half days (Collen, Schetz et al. 1977). The amino acid sequence of AT is well-conserved and the homology among cow, sheep, rabbit, mouse and human is 84%-89% (Olson and Bjork 1994). Although the primary physiological targets of AT are thrombin and FXa, AT also inhibits FIXa, FXla, FXIla, as well as FVIIa to a lesser extent. AT exerts its inhibition together with heparin. In presence of heparin the inhibition rate of thrombin and FXa by AT increases by 3 to 4 orders of magnitude from 7-11×10³ M⁻¹ s⁻¹ to 1.5-4×10⁷ M⁻¹ s⁻¹ and from 2.5×10³ M⁻¹ s⁻¹ to 1.25-2.5×10⁷ M⁻¹ s⁻¹, respectively (Olson, Swanson et al. 2004). Unlike TFPI and APC, which inhibit coagulation solely at the initiating stage and the amplification stage respectively, AT exerts its inhibition on coagulation at both the initiation and amplification stage. Therefore, blocking AT could have a more potent pro-coagulant effect than blocking either TFPI or APC alone.

The inventors have isolated several single-domain antibodies (sdAb) with the required properties and characterized by the complementarity determining regions (CDRs) of said sdAb and thus determined the CDRs of said sdAb (following tables):

TABLE A
Sequences of KB-AT-001 domains.

| KB-AT-001 | |
|---|---|
| domains | Sequences |
| CDR1 | SEQ ID NO: 1 |
| | GRTFRNYV |
| CDR2 | SEQ ID NO: 2 |
| | INRSGAIT |
| CDR3 | SEQ ID NO: 3 |
| | AAGETTWSIRRDDYDY |
| SEQUENCE | SEQ ID NO: 4 |
| KB-AT-001 | QVQLQQS GGDLAQRGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSS |

In particular, the invention relates to an isolated single-domain antibody (sdAb) comprising a CDR1 having at least 50% sequence identity with sequence set forth as SEQ ID NO: 1, a CDR2 having at least 50% sequence identity with sequence set forth as SEQ ID NO: 2 and a CDR3 having at least 50% sequence identity with sequence set forth as SEQ ID NO: 3.

According to the invention, a first amino acid sequence having at least 50% of identity with a second amino acid sequence means that the first amino acid sequence has 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the isolated single-domain antibody according to the invention comprises a CDR1 having a sequence set forth as SEQ ID NO: 1, a CDR2 having a sequence set forth as SEQ ID NO: 2 and a CDR3 having a sequence set forth as SEQ ID NO: 3.

In some embodiments, the isolated single-domain antibody according to the invention comprising a sequence KB-AT-001 having at least 70% sequence identity with sequence set forth as SEQ ID NO: 4.

According to the invention, a first amino acid sequence having at least 70% of identity with a second amino acid sequence means that the first amino acid sequence has 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or 100% of identity with the second amino acid sequence.

In some embodiments, the isolated single-domain antibody according to the invention comprises KB-AT-001 having a sequence set forth as SEQ ID NO: 4.

It should be further noted that the sdAb KB-AT-001 cross-reacts with rabbit, simian, rat and murine AT, which is of interest for preclinical evaluation and toxicological studies.

TABLE B
Sequences of KB-AT-002 domains.

| KB-AT-002 | |
|---|---|
| domains | Sequences |
| CDR1 | SEQ ID NO: 5 |
| | SGRTFNNNG |
| CDR2 | SEQ ID NO: 6 |
| | ISWSGGST |
| CDR3 | SEQ ID NO: 7 |
| | AARTRYNSGLFSRNYDY |
| SEQUENCE | SEQ ID NO: 8 |
| KB-AT-002 | QVQLVQSGGGLVQAGGSLRLSCAASGRTFNNNGMGWFRQAPGKEREFV |
| | AAISWSGGSTYYADSVKGRYIMSRDNAKNTVYLQMNSLKPEDTAVYYC |
| | AARTRYNSGLFSRNYDYWGQGTQVTVSS |

In particular, the invention relates to an isolated single-domain antibody (sdAb) comprising a CDR1 having at least 50% sequence identity with sequence set forth as SEQ ID NO:5, a CDR2 having at least 50% sequence identity with sequence set forth as SEQ ID NO: 6 and a CDR3 having at least 50% sequence identity with sequence set forth as SEQ ID NO:7.

In some embodiments, the isolated single-domain antibody according to the invention comprises a CDR1 having a sequence set forth as SEQ ID NO:5, a CDR2 having a sequence set forth as SEQ ID NO: 6 and a CDR3 having a sequence set forth as SEQ ID NO:7.

In some embodiments, the isolated single-domain antibody according to the invention comprising a sequence KB-AT-002 having at least 70% identity with sequence set forth as SEQ ID NO:8.

In some embodiments, the isolated single-domain antibody according to the invention comprises KB-AT-002 having a sequence set forth as SEQ ID NO: 8

It should be further noted that the sdAb KB-AT-002 cross-reacts with rabbit, canine, simian, bovine, porcine, rat and murine AT, which is of interest for preclinical evaluation and toxicological studies.

TABLE C
Sequences of KB-AT-003 domains.

| KB-AT-003 | |
|---|---|
| domains | Sequences |
| CDR1 | SEQ ID NO: 9 |
| | ALTFSSRAW |
| CDR2 | SEQ ID NO: 10 |
| | ITGGGTTN |
| CDR3 | SEQ ID NO: 11 |
| | NGYRYTYA |
| SEQUENCE | SEQ ID NO: 12 |
| KB-AT-003 | QVQLVQSGGGLVQPGGSLRLSCAASALTFSSRAWAWYRQAPGKQRELV |
| | ASITGGGTTNYADSVKGRFTISRDNAKNTVYLQMNSLKPEDTAVHYCN |
| | GYRYTYAWGQGTQVTVSS |

In particular, the invention relates to an isolated single-domain antibody (sdAb) comprising a CDR1 having at least 50% sequence identity with sequence set forth as SEQ ID NO:9, a CDR2 having at least 50% sequence identity with sequence set forth as SEQ ID NO: 10 and a CDR3 having at least 50% sequence identity with sequence set forth as SEQ ID NO:11.

In some embodiments, the isolated single-domain antibody according to the invention comprises a CDR1 having a sequence set forth as SEQ ID NO:9, a CDR2 having a sequence set forth as SEQ ID NO: 10 and a CDR3 having a sequence set forth as SEQ ID NO:11.

In some embodiments, the isolated single-domain antibody according to the invention comprising a sequence KB-AT-003 having at least 70% sequence identity with sequence set forth as SEQ ID NO: 12.

In some embodiments, the isolated single-domain antibody according to the invention comprises KB-AT-003 having a sequence set forth as SEQ ID NO: 12

It should be further noted that the sdAb KB-AT-003 cross-reacts with canine, simian, rat and murine AT, which is of interest for preclinical evaluation and toxicological studies.

TABLE D
Sequences of KB-AT-004 domains.

| KB-AT-004 | |
|---|---|
| domains | Sequences |
| CDR1 | SEQ ID NO: 13 |
| | AMTFSIR |
| CDR2 | SEQ ID NO: 14 |
| | IGTGDIT |
| CDR3 | SEQ ID NO: 15 |
| | NGYRSTYA |
| SEQUENCE | SEQ ID NO: 16 |
| KB-AT-004 | VQLQQSGGGLVQPGGSLRLSCAASAMTFSIRAWAWYRQAPG |
| | KQRELVASIGTGDITNYADSVKGRFTISRDNAKNTFYLQM |
| | NSLKPEDTAVYYCNGYRSTYAWGQGTQVTVSS |

In particular, the invention relates to an isolated single-domain antibody (sdAb) comprising a CDR1 having at least 50%, sequence identity with sequence set forth as SEQ ID NO:13, a CDR2 having at least 50%, sequence identity with sequence set forth as SEQ ID NO: 14 and a CDR3 having at least 50%, sequence identity with sequence set forth as SEQ ID NO:15.

In some embodiments, the isolated single-domain antibody according to the invention comprises a CDR1 having a sequence set forth as SEQ ID NO: 13, a CDR2 having a sequence set forth as SEQ ID NO: 14 and a CDR3 having a sequence set forth as SEQ ID NO: 15.

In some embodiments, the isolated single-domain antibody according to the invention comprising a sequence KB-AT-004 having at least 70% sequence identity with sequence set forth as SEQ ID NO: 16.

In some embodiments, the isolated single-domain antibody according to the invention comprises KB-AT-004 having a sequence set forth as SEQ ID NO: 16.

It should be further noted that the sdAb KB-AT-004 cross-reacts with canine, simian, porcine, rat and murine AT, which is of interest for preclinical evaluation and toxicological studies.

TABLE E
Sequences of KB-AT-005 domains.

| KB-AT-005 | |
|---|---|
| domains | Sequences |
| CDR1 | SEQ ID NO: 17 |
| | GRDFNDAAL |
| CDR2 | SEQ ID NO: 18 |
| | ITSGGVR |
| CDR3 | SEQ ID NO: 19 |
| | KADSFKGDYDTSWYLY |
| SEQUENCE | SEQ ID NO: 20 |
| KB-AT-005 | EVQLVESGGGLVQPGGSLRLSCEASGRDFNDAALGWSRQVPGKARETV |
| | AMITSGGVRNYAETVKDRFTISRDNAKNTVYLDMNNLQPDDTGVYYCK |
| | ADSFKGDYDTSWYLYWGQGTQVTVSS |

In particular, the invention relates to an isolated single-domain antibody (sdAb) comprising a CDR1 having at least 50%, sequence identity with sequence set forth as SEQ ID NO:17, a CDR2 having at least 50 sequence identity with sequence set forth as SEQ ID NO: 18 and a CDR3 having at least 50%, sequence identity with sequence set forth as SEQ ID NO:19.

In some embodiments, the isolated single-domain antibody according to the invention comprises a CDR1 having a sequence set forth as SEQ ID NO: 17, a CDR2 having a sequence set forth as SEQ ID NO: 18 and a CDR3 having a sequence set forth as SEQ ID NO: 19.

In some embodiments, the isolated single-domain antibody according to the invention comprising a sequence KB-AT-005 having at least 70% sequence identity with sequence set forth as SEQ ID NO: 20.

In some embodiments, the isolated single-domain antibody according to the invention comprises KB-AT-005 having a sequence set forth as SEQ ID NO: 20.

It should be further noted that the sdAb KB-AT-005 cross-reacts with simian and murine AT, which is of interest for preclinical evaluation and toxicological studies.

TABLE F
Sequences of KB-AT-006 domains.

| KB-AT-006 | |
|---|---|
| domains | Sequences |
| CDR1 | SEQ ID NO: 21 |
| | GRTFSNNG |
| CDR2 | SEQ ID NO: 22 |
| | ISWSSGST |
| CDR3 | SEQ ID NO: 23 |
| | AARTRYNSGYFTRNYDY |
| SEQUENCE | SEQ ID NO: 24 |
| KB-AT-006 | QVQLQQSGGGLVQAGGSLRLSCAASGRTFSNNGMGWFRQAPGKEREFV |
| | AAISWSSGSTYYADSVKGRYTISRDNAKNTVYLQMNSLKPEDTAVYYCA |
| | ARTRYNSGYFTRNYDYWGQGTQVTVSS |

In particular, the invention relates to an isolated single-domain antibody (sdAb) comprising a CDR1 having at least 50%, sequence identity with sequence set forth as SEQ ID NO:21, a CDR2 having at least 50%, sequence identity with sequence set forth as SEQ ID NO: 22 and a CDR3 having at least 50%, sequence identity with sequence set forth as SEQ ID NO:23.

In some embodiments, the isolated single-domain antibody according to the invention comprises a CDR1 having a sequence set forth as SEQ ID NO: 21, a CDR2 having a sequence set forth as SEQ ID NO: 22 and a CDR3 having a sequence set forth as SEQ ID NO: 23.

In some embodiments, the isolated single-domain antibody according to the invention comprising a sequence KB-AT-006 having at least 70% with sequence set forth as SEQ ID NO: 24.

In some embodiments, the isolated single-domain antibody according to the invention comprises KB-AT-006 having a sequence set forth as SEQ ID NO: 24.

It should be further noted that the sdAb KB-AT-006 cross-reacts with rabbit, canine, simian, porcine, rat and murine AT, which is of interest for preclinical evaluation and toxicological studies.

TABLE G
Sequences of KB-AT-007 domains.

| KB-AT-007 | |
|---|---|
| domains | Sequences |
| CDR1 | SEQ ID NO: 25 |
| | GRTFRNYV |
| CDR2 | SEQ ID NO: 26 |
| | INRSGAIT |
| CDR3 | SEQ ID NO: 27 |
| | AAGETTWSIRRDDYDY |
| SEQUENCE | SEQ ID NO: 28 |
| KB-AT-007 | QVQLQQSGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSS |

In particular, the invention relates to an isolated single-domain antibody (sdAb) comprising a CDR1 having at least 50%, sequence identity with sequence set forth as SEQ ID NO:25, a CDR2 having at least 50%, sequence identity with sequence set forth as SEQ ID NO: 26 and a CDR3 having at least 50% sequence identity with sequence set forth as SEQ ID NO:27.

In some embodiments, the isolated single-domain antibody according to the invention comprises a CDR1 having a sequence set forth as SEQ ID NO: 25, a CDR26 having a sequence set forth as SEQ ID NO: 22 and a CDR3 having a sequence set forth as SEQ ID NO: 27.

In some embodiments, the isolated single-domain antibody according to the invention comprising a sequence KB-AT-007 having at least 70% sequence identity with sequence set forth as SEQ ID NO: 28.

In some embodiments, the isolated single-domain antibody according to the invention comprises KB-AT-007 having a sequence set forth as SEQ ID NO: 28.

It should be further noted that the sdAb KB-AT-007 cross-reacts with simian and murine AT, which is of interest for preclinical evaluation and toxicological studies.

In some embodiments, the single domain antibody is a “humanized” single-domain antibody. As used herein the term “humanized” refers to a single-domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favorable properties of single-domain antibodies of the invention. The one skilled in the art is able to determine and select suitable humanizing substitutions or suitable combinations of humanizing substitutions.

In a second aspect, the invention relates to a drug conjugate comprising the isolated single domain antibody of the present invention linked to a heterologous moiety.

In some embodiments, the heterologous moiety is an aptamer, a nucleic acid, a polypeptide, another single domain antibody or a therapeutic polypeptide.

In some embodiments, the single domain antibody of the present invention is conjugated to the heterologous moiety. As used herein, the term “conjugation” has its general meaning in the art and means a chemical conjugation. Techniques for conjugating heterologous moiety to polypeptides, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. In particular the one skilled in the art can also envisage a polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site—specifically conjugated to the Fc-containing polypeptide through the acyl donor glutamine-containing tag or the accessible/exposed/reactive endogenous glutamine (WO 2012059882). The term “transglutaminase”, used interchangeably with “TGase” or “TG”, refers to an enzyme capable of cross-linking proteins through an acyl-transfer reaction between the γ-carboxamide group of peptide-bound glutamine and the r-amino group of a lysine or a structurally related primary amine such as amino pentyl group, e.g. a peptide-bound lysine, resulting in a ε-(γ-glutamyl) lysine isopeptide bond. TGases include, inter alia, bacterial transglutaminase (BTG) such as the enzyme having EC reference EC 2.3.2.13 (protein-glutamine-γ-glutamyltransferase). In some embodiments, the single domain antibody of the present invention is conjugated to the heterologous moiety by a linker molecule. As used herein, the term “linker molecule” refers to any molecule attached to the single domain antibody of the present invention. The attachment is typically covalent. In some embodiments, the linker molecule is flexible and does not interfere with the binding of the single domain antibody of the present invention.

In some embodiments, when the heterologous moiety is a heterologous polypeptide, the single domain antibody of the present invention is fused to the heterologous polypeptide to form a fusion protein.

A “fusion” or “chimeric” protein or polypeptide comprises a first amino acid sequence linked to a second amino acid sequence with which it is not naturally linked in nature. The amino acid sequences which normally exist in separate proteins can be brought together in the fusion polypeptide. A fusion protein is created, for example, by chemical synthesis, or by creating and translating a polynucleotide in which the polypeptide regions are encoded in the desired relationship.

According to the invention, the fusion protein comprises at least one isolated single domain antibody (sbAb) according to the invention that is fused either directly or via a spacer at its C-terminal end to the N-terminal end of the heterologous polypeptide, or at its N-terminal end to the C-terminal end of the heterologous polypeptide. As used herein, the term “directly” means that the (first or last) amino acid at the terminal end (N or C-terminal end) of the the single domain antibody is fused to the (first or last) amino acid at the terminal end (N or C-terminal end) of the heterologous polypeptide. In other words, in this embodiment, the last amino acid of the C-terminal end of said sdAb is directly linked by a covalent bond to the first amino acid of the N-terminal end of said heterologous polypeptide, or the first amino acid of the N-terminal end of said sdAb is directly linked by a covalent bond to the last amino acid of the C-terminal end of said heterologous polypeptide. As used herein, the term “spacer” also called “linker” refers to a sequence of at least one amino acid that links the sdAb of the invention to the heterologous polypeptide. Such a spacer may be useful to prevent steric hindrances. Examples of linkers disclosed in the present invention have the following sequences (Gly3-Ser)4, (Gly3-Ser), Ser-Gly or (Ala-Ala-Ala).

In some embodiments the heterologous moiety is another single domain antibody of the present invention. According to the invention, the drug conjugates can thus comprise a sole single-domain antibody as referred to herein as “monovalent” drug conjugate. Drug conjugates that comprise or essentially consist of two or more single-domain antibodies according to the invention are referred to herein as “multivalent” polypeptides. Typically, multivalent polypeptides could be: biparatopic antibody, trivalent antibody or quadrivalent antibody.

In some embodiments, the fusion protein is a biparatopic polypeptide. As used herein, the term “biparatopic” polypeptide means a polypeptide comprising a single domain antibody and a second single domain antibody as herein defined, wherein these two single domain antibodies are capable of binding to two different epitopes of one antigen (e.g. antithrombin), which epitopes are not normally bound at the same time by one monospecific immunoglobulin, such as e.g. a conventional antibody or one single domain antibody. Biparatopic polypeptide is also called as bivalent antibody.

In some embodiments, the two single domain antibodies of the biparatopic polypeptide of the present invention can be linked to each other directly (i.e. without use of a linker) or via a linker. The linker is typically a linker peptide and will, according to the invention, be selected so as to allow binding of the two single domain antibodies to each of their at least two different epitopes of antithrombin. Suitable linkers inter alia depend on the epitopes and, specifically, the distance between the epitopes on antithrombin to which the single domain antibodies bind, and will be clear to the skilled person based on the disclosure herein, optionally after some limited degree of routine experimentation. Also, the two single domain antibodies that bind to antithrombin may also be linked to each other via a third single domain antibody (in which the two single domain antibodies may be linked directly to the third domain antibody or via suitable linkers). Such a third single domain antibody may for example be a single domain antibody that provides an increased half-life. For example, the latter single domain antibody may be a single domain antibody that is capable of binding to a (human) serum protein such as (human) serum albumin or (human) transferrin, as further described herein. In some embodiments, two or more single domain antibodies that bind to antithrombin are linked in series (either directly or via a suitable linker) and the third (single) single domain antibody (which may provide for increased half-life, as described above) is connected directly or via a linker to one of these two or more aforementioned single domain antibodies. Suitable linkers are described herein in connection with specific polypeptides of the invention and may—for example and without limitation—comprise an amino acid sequence, which amino acid sequence preferably has a length of 9 or more amino acids, more preferably at least 17 amino acids, such as about 20 to 40 amino acids. However, the upper limit is not critical but is chosen for reasons of convenience regarding e.g. biopharmaceutical production of such polypeptides. The linker sequence may be a naturally occurring sequence or a non-naturally occurring sequence. If used for therapeutical purposes, the linker is preferably non-immunogenic in the subject to which the anti-antithrombin polypeptide of the invention is administered. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)4, (gly3ser)4 and Ser-Gly.

In some embodiments, the heterologous moiety is a single domain antibody according to the invention. Accordingly, the fusion protein comprises at least one single domain antibody as described above.

In a particular embodiment, the fusion protein is a biparatopic antibody. Typically, the fusion protein comprises two single domains antibodies. For example, a first single domain antibody is directly linked to another single domain antibody with which it is not naturally linked in nature via linker.

In a particular embodiment, the invention relates to a biparatopic antibody, which comprises a KB-AT-002 derivative as defined above and a KB-AT-003 derivative as defined above. This biparatopic antibody has the following sequence:

TABLE H
Sequence of KB-AT-002/003 domains.

| KB-AT-002/003 | Sequence |
|---|---|
| SEQUENCE KB- | SEQ ID NO: 29 |
| AT-002/003 | QVQLQESGGGLVQAGGSLRLSCAASGRTFNNNGMGWFRQAPGKEREF |
| | VAAISWSGGSTYYADSVKGRYIMSRDNAKNTVYLQMNSLKPEDTAVY |
| | YCAARTRYNSGLFSRNYDYWGQGTQVTVSSGGGSGGGSGGGSGGGSQ |
| | VQLQESGGGLVQPGGSLRLSCAASALTFSSRAWAWYRQAPGKQRELVA |
| | SITGGGTTNYADSVKGRFTISRDNAKNTVYLQMNSLKPEDTAVHYCNG |
| | YRYTYAWGQGTQVTVSS |
| | Italic: (GGGS)4 linker sequence |

In some embodiments, the fusion protein according to the invention comprising the sequences KB-AT-002 and KB-AT-003 having at least 70% sequence identity with sequence set forth as SEQ ID NO: 29 In some embodiments, the fusion protein according to the invention comprises KB-AT-002/003 having a sequence set forth as SEQ ID NO: 29.

In a particular embodiment, the invention relates to a biparatopic antibody which comprises isolated single domain antibody KB-AT-001 as described above which is linked to the isolated single domain antibody KB-AT-002 as described above. This biparatopic antibody has the following sequence:

TABLE I
Sequence of KB-AT-001/002 domains.

| KB-AT-001/002 | Sequence |
|---|---|
| SEQUENCE KB- | SEQ ID NO: 30 |
| AT-001/002 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLV |
| | QSGGGLVQAGGSLRLSCAASGRTFNNNGMGWFRQAPGKEREFVAAIS |
| | WSGGSTYYADSVKGRYIMSRDNAKNTVYLQMNSLKPEDTAVYYCAAR |
| | TRYNSGLFSRNYDYWGQGTQVTVSS |
| | Italic: (GGGS)4 linker sequence |

In some embodiments, the fusion protein according to the invention comprising the sequences KB-AT-001 and KB-AT-002 having at least 70% sequence identity with sequence set forth as SEQ ID NO: 30 In some embodiments, the fusion protein according to the invention comprises KB-AT-001/002 having a sequence set forth as SEQ ID NO: 30.

In a particular embodiment, the invention relates to a biparatopic antibody which comprises isolated single domain antibody KB-AT-001 as described above which is linked to the isolated single domain antibody KB-AT-003 as described above. This biparatopic antibody has the following sequence:

TABLE J
Sequence of KB-AT-001/003 domains.

| KB-AT-001/003 | Sequence |
|---|---|
| SEQUENCE KB- | SEQ ID NO: 31 |
| AT-001/003 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLQ |
| | ESGGGLVQPGGSLRLSCAASALTESSRAWAWYRQAPGKQRELVASITG |
| | GGTTNYADSVKGRFTISRDNAKNTVYLQMNSLKPEDTAVHYCNGYRY |
| | TYAWGQGTQVTQVTVSS |
| | Italic: (GGGS)4 linker sequence |

In some embodiments, the fusion protein according to the invention comprising the sequences KB-AT-001 and KB-AT-003 having at least 70% sequence identity with sequence set forth as SEQ ID NO: 31 In some embodiments, the fusion protein according to the invention comprises KB-AT-001/003 having a sequence set forth as SEQ ID NO: 31.

In a particular embodiment, the invention relates to a biparatopic antibody which comprises isolated single domain antibody KB-AT-001 as described above which is linked to the isolated single domain antibody KB-AT-005 as described above. This biparatopic antibody has the following sequence:

TABLE K
Sequence of KB-AT-001/005 domains.

| KB-AT-001/005 | Sequence |
|---|---|
| SEQUENCE KB- | SEQ ID NO: 32 |
| AT-001/005 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSEVQLVE |
| | SGGGLVQPGGSLRLSCEASGRDFNDAALGWSRQVPGKARETVAMITSG |
| | GVRNYAETVKDRFTISRDNAKNTVYLDMNNLQPDDTGVYYCKADSFK |
| | GDYDTSWYLYWGQGTQVTVSS |
| | Italic: (GGGS)4 linker sequence |

In some embodiments, the fusion protein according to the invention comprising the sequences KB-AT-001 and KB-AT-005 having at least 70% sequence identity with sequence set forth as SEQ ID NO: 32 In some embodiments, the fusion protein according to the invention comprises KB-AT-001/005 having a sequence set forth as SEQ ID NO: 32.

In a particular embodiment, the fusion protein is a trivalent antibody. Typically, the fusion protein comprises two single domains antibodies which are linked via two linkers.

In a particular embodiment, the fusion protein a trivalent antibody which comprises two isolated single domain antibodies KB-AT-001 according to the invention, which are linked to the isolated single domain antibody KB-AT-002 according to the invention. This trivalent antibody has the following sequence:

TABLE L
Sequence of KB-AT-112

| KB-AT-112 | Sequence |
|---|---|
| SEQUENCE KB- | SEQ ID NO: 33 |
| AT-112 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLQ |
| | ESGGGLVQAGGSLRLSCAASGRTFRNYVMGWFRQAPGKDPEFIAGINRS |
| | GAITYYGDSVKGRFTISRDNAKNTVSLQMNSLEPEDTAVYYCAAGETT |
| | WSIRRDDYDYWGQGTQVTVSSGGGSGGGSGGGSGGGSQVQLVQSGGG |
| | LVQAGGSLRLSCAASGRTFNNNGMGWFRQAPGKEREFVAAISWSGGST |
| | YYADSVKGRYIMSRDNAKNTVYLQMNSLKPEDTAVYYCAARTRYNSG |
| | LFSRNYDYWGQGTQVTVSS |
| | Italic: (GGGS)4 linker sequence |

In some embodiments, the fusion protein according to the invention comprising two sequences KB-AT-001 and one KB-AT-002 sequence having at least 70% sequence identity with sequence set forth as SEQ ID NO: 33.

In some embodiments, the fusion protein according to the invention comprises KB-AT-112 having a sequence set forth as SEQ ID NO: 33.

In a particular embodiment, the invention relates to a trivalent antibody which comprises two isolated single domain antibodies KB-AT-001 according to the invention, which are linked to the isolated single domain antibody KB-AT-003 according to the invention. This trivalent antibody has the following sequence:

TABLE M
Sequence of KB-AT-113

| KB-AT-113 | Sequence |
|---|---|
| SEQUENCE | SEQ ID NO: 34 |
| KB-AT-113 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLQ |
| | ESGGGLVQAGGSLRLSCAASGRTFRNYVMGWFRQAPGKDPEFIAGINRS |
| | GAITYYGDSVKGRFTISRDNAKNTVSLQMNSLEPEDTAVYYCAAGETT |
| | WSIRRDDYDYWGQGTQVTVSSGGGSGGGSGGGSGGGSQVQLQESGGG |
| | LVQPGGSLRLSCAASALTFSSRAWAWYRQAPGKQRELVASITGGGTTN |
| | YADSVKGRFTISRDNAKNTVYLQMNSLKPEDTAVHYCNGYRYTYAWG |
| | QGTQVTQVTVSS |
| | Italic: (GGGS)4 linker sequence |

In some embodiments, the fusion protein according to the invention comprising two sequences KB-AT-001 and one KB-AT-003 sequence having at least 70% sequence identity with sequence set forth as SEQ ID NO: 34

In some embodiments, the fusion protein according to the invention comprises KB-AT-113 having a sequence set forth as SEQ ID NO: 34.

In a particular embodiment, the invention relates to a trivalent antibody which comprises two isolated single domain antibodies KB-AT-001 according to the invention, which are linked to the isolated single domain antibody KB-AT-005 according to the invention. This trivalent antibody has the following sequence:

TABLE N
Sequence of KB-AT-115

| KB-AT-115 | Sequence |
|---|---|
| SEQUENCE | SEQ ID NO: 35 |
| KB-AT-115 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLQ |
| | ESGGGLVQAGGSLRLSCAASGRTFRNYVMGWFRQAPGKDPEFIAGINRS |
| | GAITYYGDSVKGRFTISRDNAKNTVSLQMNSLEPEDTAVYYCAAGETT |
| | WSIRRDDYDYWGQGTQVTVSSGGGSGGGSGGGSGGGSEVQLVESGGG |
| | LVQPGGSLRLSCEASGRDFNDAALGWSRQVPGKARETVAMITSGGVRN |
| | YAETVKDRFTISRDNAKNTVYLDMNNLQPDDTGVYYCKADSFKGDYD |
| | TSWYLYWGQGTQVTVSS |
| | Italic: (GGS)4 linker sequence |

In some embodiments, the fusion protein according to the invention comprising two sequences KB-AT-001 and one KB-AT-005 sequence having at least 70% sequence identity with sequence set forth as SEQ ID NO: 35.

In some embodiments, the fusion protein according to the invention comprises KB-AT-115 having a sequence set forth as SEQ ID NO: 35.

In a particular embodiment, the fusion protein is a quadrivalent antibody. Typically, the fusion protein comprises four single domains antibodies which are linked each other via three linkers.

In a particular embodiment, the fusion protein is a quadrivalent antibody which comprises two isolated single domain antibodies KB-AT-001 according to the invention, which are linked to the isolated single domain antibody KB-AT-002 according to the invention which is linked to the single domain antibody KB-AT-003. This quadrivalent antibody has the following sequence:

TABLE O
Sequence of KB-AT-1123

| KB-AT-1123 | Sequence |
|---|---|
| SEQUENCE | SEQ ID NO: 36 |
| KB-AT-1123 | QVQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYCA |
| | AGETTWSIRRDDYDYWGQGTQVTVSSGGGSGGGSGGGSGGGSQVQLQ |
| | ESGGGLVQAGGSLRLSCAASGRTFRNYVMGWFRQAPGKDPEFIAGINRS |
| | GAITYYGDSVKGRFTISRDNAKNTVSLQMNSLEPEDTAVYYCAAGETT |
| | WSIRRDDYDYWGQGTQVTVSSGGGSGGGSGGGSGGGSQVQLVQSGGG |
| | LVQAGGSLRLSCAASGRTFNNNGMGWFRQAPGKEREFVAAISWSGGST |
| | YYADSVKGRYIMSRDNAKNTVYLQMNSLKPEDTAVYYCAARTRYNSG |
| | LFSRNYDYWGQGTQVTVSSGGGSGGGSGGGSGGGSQVQLQESGGGLV |
| | QPGGSLRLSCAASALTFSSRAWAWYRQAPGKQRELVASITGGGTTNYA |
| | DSVKGRFTISRDNAKNTVYLQMNSLKPEDTAVHYCNGYRYTYAWGQG |
| | TQVTQVTVSS |
| | Italic: (GGGS)4 linker sequence |

In some embodiments, the fusion protein according to the invention comprising two sequences KB-AT-001, one KB-AT-002 sequence and one KB-AT-003 sequence having at least 70% sequence identity with sequence set forth as SEQ ID NO: 36.

In some embodiments, the fusion protein according to the invention comprises KB-AT-1123 having a sequence set forth as SEQ ID NO: 36.

In some embodiments, the heterologous moiety is a polypeptide. Typically, the single domains antibodies or multivalent antibodies according to the invention are linked to a polypeptide such as albumin, an albumin-binding peptide, VWF or a fragment thereof, or a C4BP-derived polypeptide.

In a particular embodiment, the fusion protein comprises a biparatopic antibody as described above which is linked to VWF A1 domain. Typically, the biparatopic antibody is KB-AT-002/003 is linked to VWF A1 domain sequence. Such fusion protein has the following sequence:

TABLE P
Sequence of VWF-A1/KB-AT-002/003

| VWF-A1/KB- | |
|---|---|
| AT-002/003 | Sequence |
| SEQUENCE | SEQ ID NO: 37 |
| VWF-A1/KB- | QVQLQESGGGLVQAGGSLRLSCAASGRTFNNNGMGWFRQAPGKEREF |
| AT-002/003 | VAAISWSGGSTYYADSVKGRYIMSRDNAKNTVYLQMNSLKPEDTAVY |
| | YCAARTRYNSGLFSRNYDYWGQGTQVTVSSGGGSGGGSGGGSGGGSQ |
| | VQLQESGGGLVQPGGSLRLSCAASALTFSSRAWAWYRQAPGKQRELVA |
| | SITGGGTTNYADSVKGRFTISRDNAKNTVYLQMNSLKPEDTAVHYCNG |
| | YRYTYAWGQGTQVTVSSGRGGGSGGGSGGGSDISEPPLHDFYCSRLLD |
| | LVFLLDGSSRLSEAEFEVLKAFVVDMMERLRISQKWVRVAVVEYHD |
| | GSHAYIGLKDRKRPSELRRIASQVKYAGSQVASTSEVLAYTLFQIFSK |
| | IDRPEASRIALLLMASQEPQRMSRNFVRYVQGLKKKKVIVIPVGIGP |
| | HANLKQIRLIEKQAPENKAFVLSSVDELEQQRDEIVSYLCDLAPEAP |
| | PPTLPPDMAQVTV |
| | Italic: (GGGS)4 linker sequence |
| | In bold: Sequence of human VWF A1 domain |

In some embodiments, the fusion protein according to the invention comprising one sequence of KB-AT-002, one sequence of KB-AT-003 and sequence of human VWF A1 domain, having at least 70% sequence identity with sequence set forth as SEQ ID NO: 37.

In some embodiments, the fusion protein according to the invention comprises VWF-A1/KB-AT-002/003 having a sequence set forth as SEQ ID NO: 37.

In a particular embodiment, the polypeptide heterologous is a polypeptide derived from C4BP.

As used herein, the term “C4BP” refers to C4b-binding protein which is a protein involved in the complement system where it acts as inhibitor. C4BP has an octopus-like structure with a central stalk and seven branching alpha-chains. The main form of C4BP in human blood is composed of 7 identical alpha-chains and one unique beta-chain, which in turn binds anticoagulant, vitamin K-dependent protein S. C4BP is a large glycoprotein (500 kDa) with an estimated plasma concentration of 200 micrograms/mL synthesized mainly in the liver.

In a particular embodiment, the fusion protein comprises an isolated single domain antibody according to the invention, which is fused with a C4BP sequence. Typically, the single domain antibody KB-AT-002 is linked to C4BP. Such protein has the following sequence:

TABLE Q
Sequence of KB-AT-002/C4BP

| KB-AT- | |
|---|---|
| 002/C4BP | Sequence |
| SEQUENCE | SEQ ID NO: 38 |
| KB-AT- | MVPARFAGVLLALALILPGTLCQVQLVQSGGGLVQAGGSLRLSCAAS |
| 002/C4BP | GRTFNNNGMGWFRQAPGKEREFVAAISWSGGSTYYADSVKGRYIM |
| | SRDNAKNTVYLQMNSLKPEDTAVYYCAARTRYNSGLFSRNYDYWG |
| | QGTQVTVSSSGETPEGCEQVLTGKRLMQCLPNPEDVKMALEVYKLSLEIE |
| | QLELQRDSARQSTLDKELEDQVDPRLIDGK |
| | Underlined: signal peptide; Bold: KB-AT-002; Bold underlined: |
| | short linker peptide Ser-Gly; |
| | Italic-underlined: 57 amino acids corresponding to human |
| | C4BP residues 541-597; |
| | EDQVDPRLIDGK: epitope for antibody HPC4 |

In some embodiments, the fusion protein according to the invention comprising one KB-AT-002 and sequence of human C4BP, having at least 70% sequence identity with sequence set forth as SEQ ID NO: 38.

In some embodiments, the fusion protein according to the invention comprises KB-AT-002-C4BP having a sequence set forth as SEQ ID NO: 38.

In some embodiments, the single domain antibody KB-AT-003 is linked to C4BP. Such protein has the following sequence:

TABLE R
Sequence of KB-AT-003/C4BP

| KB-AT- | |
|---|---|
| 003/C4BP | Sequence |
| SEQUENCE | SEQ ID NO: 39 |
| KB-AT-003/- | MVPARFAGVLLALALILPGTLCQVQLQQSGGGLVQPGGSLRLSCAAS |
| C4BP | ALTFSSRAWAWYRQAPGKQRELVASITGGGTTNYADSVKGRFTISR |
| | DNAKNTVYLQMNSLKPEDTAVHYCNGYRYTYAWGQGTQVTVSSSG |
| | ETPEGCEQVLTGKRLMQCLPNPEDVKMALEVYKLSLEIEQLELQRDSARQST |
| | LDKELEDQVDPRLIDGK |
| | Underlined: signal peptide; Bold: KB-AT-002; Bold |
| | underlined: short linker peptide Ser-Gly; |
| | Italic-underlined: 57 amino acids corresponding to human |
| | C4BP residues 541-597; |
| | EDQVDPRLIDGK: epitope for antibody HPC4 |

In some embodiments, the fusion protein according to the invention comprising one KB-AT-003 and sequence of human C4BP, having at least 70% sequence identity with sequence set forth as SEQ ID NO: 39.

In some embodiments, the fusion protein according to the invention comprises KB-AT-003-C4BP having a sequence set forth as SEQ ID NO: 39.

In some embodiments, the fusion protein comprises a biparatopic antibody as described above which is linked to a murine FVII variant. Typically, the biparatpoic antibody is KB-AT-002/003 which is linked to a murine FVII variant sequence. Such fusion protein has the following sequence:

TABLE S
Sequence of mFVII-AT-0203

| mFVII-AT-0203 | Sequence |
|---|---|
| SEQUENCE | SEQ ID NO: 40 |
| mFVII-AT-0203 | MVPQAHGLLLLCFLLQLQGPLGTAVFITQEEAHGVLHRQRRANSLLEEL |
| | WPGSLERECNEEQCSFEEAREIFKSPERTKQFVVIVYSDGDQCASNPCQN |
| | GGTCQDHLKSYVCFCLLDFEGRNCEKSKNEQLICANENGDCDQYCRDH |
| | VGTKRTCSCHEDYTLQPDEVSCKPKVEYPCGRIPVVEKRNSSSRQGRRK |
| | RRKRLVGGNVCPKGECPWQAVLKINGLLLCGAVLLDARWIVTAAHCF |
| | DNIRYWGNITVVMGEHDFSEKDGDEQVRRVTQVIMPDKYIRGKINHDIA |
| | LLRLHRPVTFTDYVVPLCLPEKSFSENTLARIRFSRVSGWGQLLDRGATA |
| | LELMSIEVPRLMTQDCLEHAKHSSNTPKITENMFCAGYMDGTKDACKG |
| | DSGGPHATHYHGTWYLTGVVSWGEGCAAIGHIGVYTRVSQYIDWLVR |
| | HMDSKLQVGVFRLPLLLTPRGVRLGGGSQVQLQESGGGLVQAGGSLRLS |
| | CAASGRTFNNNGMGWFRQAPGKEREFVAAISWSGGSTYYADSVKGRYIMSR |
| | DNAKNTVYLQMNSLKPEDTAVYYCAARTRYNSGLFSRNYDYWGQGTQVTVSS |
| | GGGSGGGSGGGSGGGSQVQLQESGGGLVQPGGSLRLSCAASALTFSSRA |
| | WAWYRQAPGKQRELVASITGGGTTNYADSVKGRFTISRDNAKNTVYLQMNSL |
| | KPEDTAVHYCNGYRYTYAWGQGTQVTVSSGGGSEDQVDPRLIDGK |
| | Thrombin-cleavage site: LTPRGVRL |
| | Linker sequences: GGGS & GGGSGGGSGGGSGGGS |
| | KB-AT-02: |
| | QVQLQESGGGLVQAGGSLRLSCAASGRTFNNNGMGWFRQAPGKEREFVAA |
| | ISWSGGSTYYADSVKGRYIMSRDNAKNTVYLQMNSLKPEDTAVYYCAARTRYN |
| | SGLFSRNYDYWGQGTQVTVSS |
| | KB-AT-03: |
| | QVQLQESGGGLVQPGGSLRLSCAASALTFSSRAWAWYRQAPGKQRELVASIT |
| | GGGTTNYADSVKGRFTISRDNAKNTVYLQMNSLKPEDTAVHYCNGYRYTYA |
| | WGQGTQVTVSS |
| | HPC4-tag for purification: EDQVDPRLIDGK |

In some embodiments, the fusion protein according to the invention comprising one KB-AT-002 sequence, one KB-AT-003 sequence and a sequence of murine FVII, having at least 70% sequence identity with sequence set forth as SEQ ID NO:40.

In some embodiments, the fusion protein according to the invention comprises mFVII-AT-0203 having a sequence set forth as SEQ ID NO: 40.

In some embodiments, the fusion protein comprises a biparatopic antibody as described above which is linked to a FVIII variant. Typically, the biparatpoic antibody is KB-AT-002/003 which is linked to a FVIII variant sequence. Such fusion protein has the following sequence:

TABLE T
Sequence of FVIII-AT-0203

| FVIII-AT-0203 | Sequence |
|---|---|
| SEQUENCE | SEQ ID NO: 41 |
| FVIII-AT-0203 | MQIELSTCFFLCLLRFCFSATRRYYLGAVELSWDYMQSDLGELPVDARF |
| | PPRVPKSFPFNTSVVYKKTLFVEFTDHLFNIAKPRPPWMGLLGPTIQAEV |
| | YDTVVITLKNMASHPVSLHAVGVSYWKASEGAEYDDQTSQREKEDDK |
| | VFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAAS |
| | ARAWPKMHTVNGYVNRSLPGLIGCHRKSVYWHVIGMGTTPEVHSIFLE |
| | GHTFLVRNHRQASLEISPITFLTAQTLLMDLGQFLLFCHISSHQHDGMEA |
| | YVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRI |
| | GRKYKKVRFMAYTDETFKTREAIQHESGILGPLLYGEVGDTLLIIFKNQA |
| | SRPYNIYPHGITDVRPLYSRRLPKGVKHLKDFPILPGEIFKYKWTVTVED |
| | GPTKSDPRCLTRYYSSFVNMERDLASGLIGPLLICYKESVDQRGNQIMSD |
| | KRNVILFSVFDENRSWYLTENIQRFLPNPAGVQLEDPEFQASNIMHSING |
| | YVFDSLQLSVCLHEVAYWYILSIGAQTDFLSVFFSGYTFKHKMVYEDTL |
| | TLFPFSGETVFMSMENPGLWILGCHNSDFRNRGMTALLKVSSCDKNTG |
| | DYYEDSYEDISAYLLSKNNAIEPRSFSGGGSQVQLQESGGGLVQAGGSLR |
| | LSCAASGRTFNNNGMGWFRQAPGKEREFVAAISWSGGSTYYADSVKGRYIMS |
| | RDNAKNTVYLQMNSLKPEDTAVYYCAARTRYNSGLFSRNYDYWGQGTQVTV |
| | SSGGGSGGGSGGGSGGGSQVQLQESGGGLVQPGGSLRLSCAASALTFSSR |
| | AWAWYRQAPGKQRELVASITGGGTTNYADSVKGRFTISRDNAKNTVYLQMN |
| | SLKPEDTAVHYCNGYRYTYAWGQGTQVTVSSGGGSEITRTTLQSDQEEIDY |
| | DDTISVEMKKEDFDIYDEDENQSPRSFQKKTRHYFIAAVERLWDYGMSS |
| | SPHVLRNRAQSGSVPQFKKVVFQEFTDGSFTQPLYRGELNEHLGLLGPYI |
| | RAEVEDNIMVTFRNQASRPYSFYSSLISYEEDQRQGAEPRKNFVKPNET |
| | KTYFWKVQHHMAPTKDEFDCKAWAYFSDVDLEKDVHSGLIGPLLVCH |
| | TNTLNPAHGRQVTVQEFALFFTIFDETKSWYFTENMERNCRAPCNIQME |
| | DPTFKENYRFHAINGYIMDTLPGLVMAQDQRIRWYLLSMGSNENIHSIH |
| | FSGHVFTVRKKEEYKMALYNLYPGVFETVEMLPSKAGIWRVECLIGEH |
| | LHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIM |
| | YSLDGKKWQTYRGNSTGTLMVFFGNVDSSGIKHNIFNPPIIARYIRLHPT |
| | HYSIRSTLRMEWMGCDLNSCSMPLGMESKAISDAQITASSYFTNMFAT |
| | WSPSKARLHLQGRSNAWRPQVNNPKEWLQVDFQKTMKVTGVTTQGV |
| | KSLLTSMYVKEFLISSSQDGHQWTLFFQNGKVKVFQGNQDSFTPVVNSL |
| | DPPLLTRYLRIHPQSWVHQIALRMEVLGCEAQDLY |
| | Linker sequences: GGGS & GGGSGGGSGGGSGGGS |
| | KB-AT-02: |
| | QVQLQESGGGLVQAGGSLRLSCAASGRTFNNNGMGWFRQAPGKEREFVAA |
| | ISWSGGSTYYADSVKGRYIMSRDNAKNTVYLQMNSLKPEDTAVYYCAARTRYN |
| | SGLFSRNYDYWGQGTQVTVSS |
| | KB-AT-03: |
| | QVQLQESGGGLVQPGGSLRLSCAASALTFSSRAWAWYRQAPGKQRELVASIT |
| | GGGTTNYADSVKGRFTISRDNAKNTVYLQMNSLKPEDTAVHYCNGYRYTYA |
| | WGQGTQVTVSS |

In some embodiments, the fusion protein according to the invention comprising one sequence of KB-AT-002, one sequence of KB-AT-003 and the sequence of human FVIII, having at least 70% sequence identity with sequence set forth as SEQ ID NO:41.

In some embodiments, the fusion protein according to the invention comprises FVIII-AT-0203 having a sequence set forth as SEQ ID NO: 41.

TABLE U
Sequence of KB-AT-114

| KB-AT-114 | Sequence |
|---|---|
| SEQUENCE | SEQ ID NO: 42 |
| KB-AT-114 | QVQLQESGGGLVQAGGSLRLSCAASGRTFRNYVMGWFRQAPGKDPEFIAGINR |
| | SGAITYYGDSVKGRFTISRDNAKNTVSLQMNSLEPEDTAVYYCAAGETTWSIRRD |
| | DYDYWGQGTQVTVSSGGGSGGGSGGGSGGGSQVQLQESGGGLVQAGGSLRL |
| | SCAASGRTFRNYVMGWFRQAPGKDPEFIAGINRSGAITYYGDSVKGRFTISRDNA |
| | KNTVSLQMNSLEPEDTAVYYCAAGETTWSIRRDDYDYWGQGTQVTVSSGGGS |
| | GGGSGGGSGGGSQVQLQSGGGLVQPGGSLRLSCAASAMTFSIRAWAWYRQAP |
| | GKQRELVASIGTGDITNYADSVKGRFTISRDNAKNTFYLQMNSLKPEDTAVYYCN |
| | GYRSTYAWGQGTQVTVSS |
| | Italic: (GGGS)4 linker sequence |

In some embodiments, the fusion protein according to the invention comprising two sequences KB-AT-001 and one KB-AT-004 sequence having at least 70% sequence identity with sequence set forth as SEQ ID NO: 42.

In some embodiments, the fusion protein according to the invention comprises KB-AT-114 having a sequence set forth as SEQ ID NO: 42.

TABLE V
Sequence of KB-AT-644

| KB-AT-644 | Sequence |
|---|---|
| SEQUENCE | SEQ ID NO: 43 |
| KB-AT-644 | QVQLQSGGGLVQAGGSLRLSCAASGRTFSNNGMGWFRQAPGKEREFVAAISW |
| | SSGSTYYADSVKGRYTISRDNAKNTVYLQMNSLKPEDTAVYYCAARTRYNSGYFT |
| | RNYDYWGQGTQVTVSSGGGSGGGSGGGSGGGSQVQLQSGGGLVQPGGSLRL |
| | SCAASAMTFSIRAWAWYRQAPGKQRELVASIGTGDITNYADSVKGRFTISRDNA |
| | KNTFYLQMNSLKPEDTAVYYCNGYRSTYAWGQGTQVTVSSGGGSGGGSGGGS |
| | GGGSQVQLQSGGGLVQPGGSLRLSCAASAMTFSIRAWAWYRQAPGKQRELVA |
| | SIGTGDITNYADSVKGRFTISRDNAKNTFYLQMNSLKPEDTAVYYCNGYRSTYAW |
| | GQGTQVTVSS |
| | Italic: (GGGS)4 linker sequence |

In some embodiments, the fusion protein according to the invention comprising one sequence KB-AT-006 and two sequences of KB-AT-004 sequence having at least 70% sequence identity with sequence set forth as SEQ ID NO: 43.

In some embodiments, the fusion protein according to the invention comprises KB-AT-644 having a sequence set forth as SEQ ID NO: 43.

TABLE W
Sequence of KB-AT-244

| KB-AT-244 | Sequence |
|---|---|
| SEQUENCE | SEQ ID NO: 44 |
| KB-AT-244 | QVQLQESGGGLVQAGGSLRLSCAASGRTFNNNGMGWFRQAPGKEREFVAAIS |
| | WSGGSTYYADSVKGRYIMSRDNAKNTVYLQMNSLKPEDTAVYYCAARTRYNSG |
| | LFSRNYDYWGQGTQVTVSSGGGSGGGSGGGSGGGSQVQLQSGGGLVQPGGS |
| | LRLSCAASAMTFSIRAWAWYRQAPGKQRELVASIGTGDITNYADSVKGRFTISRD |
| | NAKNTFYLQMNSLKPEDTAVYYCNGYRSTYAWGQGTQVTVSSGGGSGGGSGG |
| | GSGGGSQVQLQSGGGLVQPGGSLRLSCAASAMTFSIRAWAWYRQAPGKQREL |
| | VASIGTGDITNYADSVKGRFTISRDNAKNTFYLQMNSLKPEDTAVYYCNGYRSTY |
| | AWGQGTQVTVSS |
| | Italic: (GGGS)4 linker sequence |

In some embodiments, the fusion protein according to the invention comprising one sequence KB-AT-002 and two sequences of KB-AT-004 sequence having at least 70% sequence identity with sequence set forth as SEQ ID NO: 44.

In some embodiments, the fusion protein according to the invention comprises KB-AT-244 having a sequence set forth as SEQ ID NO: 44.

TABLE X
Sequence of KB-AT-443

| KB-AT-443 | Sequence |
|---|---|
| SEQUENCE | SEQ ID NO: 45 |
| KB-AT-443 | QVQLQSGGGLVQPGGSLRLSCAASAMTFSIRAWAWYRQAPGKQRELVASIGTG |
| | DITNYADSVKGRFTISRDNAKNTFYLQMNSLKPEDTAVYYCNGYRSTYAWGQGT |
| | QVTVSSGGGSGGGSGGGSGGGSQVQLQSGGGLVQPGGSLRLSCAASAMTFSIR |
| | AWAWYRQAPGKQRELVASIGTGDITNYADSVKGRFTISRDNAKNTFYLQMNSLK |
| | PEDTAVYYCNGYRSTYAWGQGTQVTVSSGGGSGGGSGGGSGGGSQVQLQESG |
| | GGLVQPGGSLRLSCAASALTFSSRAWAWYRQAPGKQRELVASITGGGTTNYADS |
| | VKGRFTISRDNAKNTVYLQMNSLKPEDTAVHYCNGYRYTYAWGQGTQVTQVTV |
| | SS |
| | Italic: (GGGS)4 linker sequence |

In some embodiments, the fusion protein according to the invention comprising two sequences of KB-AT-004 and one sequence of KB-AT-003 having at least 70% sequence identity with sequence set forth as SEQ ID NO: 45.

In some embodiments, the fusion protein according to the invention comprises KB-AT-443 having a sequence set forth as SEQ ID NO: 45.

TABLE Y
Sequence of KB-AT-002004

| KB-AT-002004 | Sequence |
|---|---|
| SEQUENCE | SEQ ID NO: 46 |
| KB-AT-002004 | QVQLQESGGGLVQAGGSLRLSCAASGRTFNNNGMGWFRQAPGKEREFVAAIS |
| | WSGGSTYYADSVKGRYIMSRDNAKNTVYLQMNSLKPEDTAVYYCAARTRYNSG |
| | LFSRNYDYWGQGTQVTVSSGGGSGGGSGGGSGGGSQVQLQSGGGLVQPGGS |
| | LRLSCAASAMTFSIRAWAWYRQAPGKQRELVASIGTGDITNYADSVKGRFTISRD |
| | NAKNTFYLQMNSLKPEDTAVYYCNGYRSTYAWGQGTQVTVTVSS |
| | Italic: (GGGS)4 linker sequence |

In some embodiments, the fusion protein according to the invention comprising one sequence of KB-AT-002 and one sequence of KB-AT-004 having at least 70% sequence identity with sequence set forth as SEQ ID NO: 46.

In some embodiments, the fusion protein according to the invention comprises KB-AT-002004 having a sequence set forth as SEQ ID NO: 46.

TABLE Z
Sequence of different construction

| SEQUENCES | SEQUENCE ID NO: |
|---|---|
| SEQUENCE | SEQ ID NO: 47 |
| KB-AT-004004 | QVQLQSGGGLVQPGGSLRLSCAASAMTFSIRAWAWYRQAPGKQRELVASIGTG |
| | DITNYADSVKGRFTISRDNAKNTFYLQMNSLKPEDTAVYYCNGYRSTYAWGQGT |
| | QVTVSSGGGSGGGSGGGSGGGSQVQLQSGGGLVQPGGSLRLSCAASAMTFSIR |
| | AWAWYRQAPGKQRELVASIGTGDITNYADSVKGRFTISRDNAKNTFYLQMNSLK |
| | PEDTAVYYCNGYRSTYAWGQGTQVTVSS |
| | Italic: (GGGS)4 linker sequence |
| SEQUENCE | SEQ ID NO: 48 |
| KB-AT-002006 | QVQLQESGGGLVQAGGSLRLSCAASGRTFNNNGMGWFRQAPGKEREFVAAIS |
| | WSGGSTYYADSVKGRYIMSRDNAKNTVYLQMNSLKPEDTAVYYCAARTRYNSG |
| | LFSRNYDYWGQGTQVTVSSGGGSGGGSGGGSGGGSQVQLQSGGGFVQAGGS |
| | LRLSCAASGRTFSNNGMGWFRQAPGKEREFVAAISWSSGSTYYADSVKGRYTISS |
| | DNAKNTVYLQMNSLKPEDTAVYYCAARTRYNRGYFTRNYDYWGQGTQVTVSS |
| | Italic: (GGGS)4 linker sequence |
| SEQUENCE | SEQ ID NO: 49 |
| KB-AT-001001 | QVQLQESGGGLVQAGGSLRLSCAASGRTFRNYVMGWFRQAPGKDPEFIAGINR |
| | SGAITYYGDSVKGRFTISRDNAKNTVSLQMNSLEPEDTAVYYCAAGETTWSIRRD |
| | DYDYWGQGTQVTVSSGGGSGGGSGGGSGGGSQVQLQESGGGLVQAGGSLRL |
| | SCAASGRTFRNYVMGWFRQAPGKDPEFIAGINRSGAITYYGDSVKGRFTISRDNA |
| | KNTVSLQMNSLEPEDTAVYYCAAGETTWSIRRDDYDYWGQGTQVTVSS |
| | Italic: (GGGS)4 linker sequence |
| SEQUENCE | SEQ ID NO: 50 |
| KB-AT-6623 | QVQLQSGGGLVQAGGSLRLSCAASGRTFSNNGMGWFRQAPGKEREFVAAISW |
| | SSGSTYYADSVKGRYTISRDNAKNTVYLQMNSLKPEDTAVYYCAARTRYNSGYFT |
| | RNYDYWGQGTQVIVSSGGGSGGGSGGGSGGGSQVQLQSGGGFVQAGGSLRL |
| | SCAASGRTFSNNGMGWFRQAPGKEREFVAAISWSSGSTYYADSVKGRYTISSDN |
| | AKNTVYLQMNSLKPEDTAVYYCAARTRYNRGYFTRNYDYWGQGTQVIVSSGG |
| | GSGGGSGGGSGGGSQVQLVQSGGGLVQAGGSLRLSCAASGRTFNNNGMGWF |
| | RQAPGKEREFVAAISWSGGSTYYADSVKGRYIMSRDNAKNTVYLQMNSLKPEDT |
| | AVYYCAARTRYNSGLFSRNYDYWGQGTQVTVSSGGGSGGGSGGGSGGGSQVQ |
| | LQESGGGLVQPGGSLRLSCAASALTFSSRAWAWYRQAPGKQRELVASITGGGTT |
| | NYADSVKGRFTISRDNAKNTVYLQMNSLKPEDTAVHYCNGYRYTYAWGQGTQV |
| | TQVTVSS |
| | Italic: (GGGS)4 linker sequence |

In some embodiments, the fusion protein according to the invention comprising two sequences of KB-AT-004 having at least 70% sequence identity with sequence set forth as SEQ ID NO: 47.

In some embodiments, the fusion protein according to the invention comprises KB-AT-004004 having a sequence set forth as SEQ ID NO: 47.

In some embodiments, the fusion protein according to the invention comprising one sequence of KB-AT-002 and one sequence of KB-AT-006 having at least 70% sequence identity with sequence set forth as SEQ ID NO: 48.

In some embodiments, the fusion protein according to the invention comprises KB-AT-002006 having a sequence set forth as SEQ ID NO: 48.

In some embodiments, the fusion protein according to the invention comprising two sequences of KB-AT-001 having at least 70% sequence identity with sequence set forth as SEQ ID NO: 49.

In some embodiments, the fusion protein according to the invention comprises KB-AT-001001 having a sequence set forth as SEQ ID NO: 49.

In some embodiments, the fusion protein according to the invention comprising two sequences of KB-AT-006, one sequence of KB-AT-002 and one sequence of KB-AT-003 having at least 70% sequence identity with sequence set forth as SEQ ID NO: 50.

In some embodiments, the fusion protein according to the invention comprises KB-AT-6623 having a sequence set forth as SEQ ID NO: 50.

In some embodiments, the heterologous moiety is a circulating protein. Typically, the single domains antibodies or multivalent antibodies according to the invention are linked to a circulating protein.

By “circulating protein”, it is meant proteins synthesized by the cells of the body organs and transported within the blood stream. Examples of circulating proteins are blood coagulation factors, proteins and hormones.

In some embodiments, the circulating protein is a therapeutic protein, i.e. a protein that can be used for the treatment of a subject. Thus, in some embodiment, the heterologous moiety is a therapeutic polypeptide, particularly having a short half-life leading to repeated administration to the patient in need thereof. Such therapeutic polypeptide may be for instance insulin, glucagon, osteoprotegerin (OPG), Angiopoietin-2 (ANGPT2), furin, growth factors or other peptide hormones.

As used herein, the term “half-life” refers to a biological half-life of a particular polypeptide in vivo. Half-life may be represented by the time required for half the quantity administered to a subject to be cleared from the circulation and/or other tissues in the animal. When a clearance curve of a given polypeptide is constructed as a function of time, the curve is usually biphasic with a rapid, α-phase and longer β-phase

In a particular embodiment, the circulating protein is a clotting factor (also referred as blood coagulation factor).

As used herein, the term “clotting factor,” refers to molecules, or analogs thereof naturally occurring or recombinant produced which prevent or decrease the duration of a bleeding episode in a subject. In other words, it means molecules having pro-clotting activity, i.e., promoting are responsible for the conversion of fibrinogen into a mesh of insoluble fibrin causing the blood to coagulate or clot. Clotting factors include factor Von Willebrand (VWF), factor VIII, vitamin K-dependent coagulation proteins (comprising factor VII, Factor IX, factor X, protein C, protein S, protein Z and prothrombin) and clotting factor V. Clotting factors of the invention may also be variants of wild-type clotting factors. The term “variants” includes insertions, deletions and substitutions, either conservative or non-conservative, where such changes do not substantially alter the active site, or active domain, which confers the biological activities of the respective clotting factor. Preferably a clotting factor is selected from the group consisting of VWF, FVII, FVIII, FIX and FX.

In a third aspect, the invention relates to a vector which comprises the single domains antibodies or drug conjugate of the present invention.

Typically the single domains antibodies or drug conjugate may be delivered in association with a vector. The single domains antibodies or drug conjugate of the present invention is included in a suitable vector, such as a plasmid, cosmid, episome, artificial chromosome, phage or a viral vector. So, a further object of the invention relates to a vector comprising a single domain antibodies or drug conjugate of the invention. Typically, the vector is a viral vector, which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g., U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein that allows transduction of cells of human and other species. Typically, the nucleic acid molecule or the vector of the present invention include “control sequences'”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In a fourth aspect, the invention relates to a method of extending or increasing half-life of a therapeutic polypeptide comprising a step of adding to the polypeptide sequence of said therapeutic polypeptide at least one sdAb directed against antithrombin or a drug conjugate which is inserted or not in to the vector according to the invention.

Typically, the single domain antibodies or multivalent antibodies of the present invention are suitable for extending or increasing the half-life of a circulating protein.

In some embodiments, the single domain antibodies according to the invention are fused to factor Von Willebrand (VWF). Particularly, the singles domains antibodies according to the invention are fused to VWF-A1 domain. Such construction corresponds to VWF-A1/KB-AT-002/003 as described above.

In some embodiments, the single domain antibodies according to the invention are fused to factor VII (FVII). Particularly, the singles domains antibodies according to the invention are fused to FVII. Such construction corresponds to FVII-AT-0203 as described above.

In some embodiments, the single domain antibodies according to the invention are fused to factor VIII (FVIII). Particularly, the singles domains antibodies according to the invention are fused to FVIII. Such construction corresponds to FVIII-AT-0203 as described above.

In a particular embodiment, the drug conjugate exhibits a reduced clearance rate and thus an extended half-life when administered to a subject, compared to a corresponding polypeptide not linked to said sdAb directed against AT and administered to said subject.

In a particular embodiment, the present invention relates to a method of extending or increasing the half-life of the single domain antibodies or the drug conjugate according to the invention which is inserted or not in to a vector.

In a further embodiment, the half-life of the single domain antibodies according to the invention can be prolonged by C4BP. Typically, the single domains antibodies according to the invention are fused to C4BP. Such construction is described above (see the fusion proteins KB-AT-002/C4BP and KB-AT-003/C4BP).

In a fifth aspect, the invention relates to a method of preventing or treating bleeding disorders in a subject in need thereof, comprising administering to said subject a therapeutically effective amount of the single domain antibody or the drug conjugate according to the invention which is inserted or not in to a vector.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. Particularly, in the present invention, the subject is a human afflicted with or susceptible to be afflicted with bleeding disorders.

The bleeding disorders that may be treated by administration of the fusion protein of the invention include, but are not limited to, hemophilia, as well as deficiencies or structural abnormalities in fibrinogen, prothrombin, Factor V, Factor VII, FIX or Factor X.

In a particular embodiment, the bleeding disorders that may be treated by administration of the fusion protein of the invention are hemophilia A or hemophilia B.

By a “therapeutically effective amount” is meant a sufficient amount of the polypeptide (or the vector containing the polypeptide) to prevent for use in a method for the treatment of bleeding disorders at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 100 mg/kg of body weight per day,

In some embodiments, the present invention relates to a method for preventing or treating heparin induced hemorrhages in a subject in need thereof, comprising administering to said subject a therapeutically effective amount of the single domain antibodies, the drug conjugate or the vector comprising the single domain antibody or drug conjugate according to the invention.

Heparin is a widely used injectable blood thinner. It is used to treat and prevent deep vein thrombosis and pulmonary embolism. Heparin is a polymer of varying chain size. Unfractionated heparin (UFH) as a pharmaceutical is heparin that has not been fractionated to sequester the fraction of molecules with low molecular weight. In contrast, low-molecular-weight heparin (LMWH) has undergone fractionation for the purpose of making its pharmacodynamics more predictable. The term “heparin induced hemorrhages” refers to the bleeding which is a major side effect of heparin when it is administered therapeutically.

In a sixth aspect, the invention relates to a pharmaceutical composition comprising the single domain antibodies or the drug conjugate according to the present invention, which is inserted or not in to a vector.

The single-domain antibodies and drug conjugate of the invention (or the vector comprising single domain antibodies or the drug conjugate) may be combined with pharmaceutically acceptable excipients, and optionally sustained-release matrices, such as biodegradable polymers, to form pharmaceutical compositions. As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The single domain antibodies or the drug conjugate (or the vector comprising single domain antibodies or the drug conjugate) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The polypeptide (or the vector containing the polypeptide) may be formulated within a therapeutic mixture to comprise about 0.0001 to 1.0 milligrams, or about 0.001 to 0.1 milligrams, or about 0.1 to 1.0 or even about 100 milligrams per dose. Multiple doses can also be administered. The invention will be further illustrated by the following figures and examples.

The invention will be further illustrated by the following figures and examples. However, these examples and figures should not be interpreted in any way as limiting the scope of the present invention.

### FIGURES

FIG. 1: Binding of human and murine antithrombin to immobilized monovalent sdAbs

Human and murine antithrombin (0-5 micromolar) were added to wells coated with monovalent sdAbs. Bound antithrombin was probed using polyclonal anti-antithrombin antibodies and detected via TMB-hydrolysis. Plotted is the observed OD at 450 nm versus the antithrombin concentration.

FIG. 2: In vivo survival of VWF A1/p.K1362A fused to KB-AT-002/003

VWF A1/p.K1362A was fused to an irrelevant sdAb (KB-UT-01) or to KB-AT-002/003 to generate VWF-A1/KB-UT-01 and VWF-A1/KB-AT-002/003, respectively. Purified proteins were given intravenously to wild-type C57B6 mice. At indicated time-points, blood was collected and residual VWF-A1 antigen was measured. Plotted is residual antigen versus time after injection. VWF-A1/KB-002/003 is removed from the circulation remarkably slower than is VWF-A1/KB-UT-01.

FIG. 3: Effect of monovalent sdAbs on thrombin activity in the presence of antithrombin

Residual amidolytic activity of thrombin towards the synthetic substrate S-2238 was measured in the absence and presence of a 10-fold molar excess antithrombin. Antithrombin was pre-incubated in the absence or presence of a 10-fold molar excess of monovalent sdAbs recognizing antithrombin. Plotted is residual thrombin activity (expressed as ΔOD/min) versus the various types of incubation mixtures. All monovalent sdAbs were able to partially (55-67%) neutralize antithrombin-mediated inhibition of thrombin.

FIG. 4: Effect of monovalent sdAbs on thrombin activity in the presence of antithrombin and unfractionated heparin

Residual amidolytic activity of thrombin towards the synthetic substrate S-2238 was measured in the absence and presence of a 10-fold molar excess antithrombin. Antithrombin was pre-incubated with heparin (1 U/ml) in the absence or presence of a 10-fold molar excess of monovalent sdAbs recognizing antithrombin. Plotted is residual thrombin activity (expressed as ΔOD/min) versus the various types of incubation mixtures. The percentage by which sdAbs were able to neutralize antithrombin-mediated inhibition of thrombin was less than 5%.

FIG. 5: Effect of monovalent sdAbs on factor Xa activity in the presence of antithrombin and low molecular weight (LMW)-heparin

Residual amidolytic activity of factor Xa towards the synthetic substrate S-2765 was measured in the absence and presence of a 10-fold molar excess antithrombin. Antithrombin was pre-incubated with LMW-heparin (1 U/ml) in the absence or presence of a 10-fold molar excess of monovalent sdAbs recognizing antithrombin. Plotted is residual factor Xa activity (expressed as ΔOD/min) versus the various types of incubation mixtures. The percentage by which sdAbs were able to neutralize antithrombin-mediated inhibition of factor Xa was less than 15%.

FIG. 6: Effect of bi-paratopic sdAbs on thrombin activity in the presence of antithrombin and unfractionated heparin

Residual amidolytic activity of thrombin towards the synthetic substrate S-2238 was measured in the absence and presence of a 10-fold molar excess antithrombin. Antithrombin was pre-incubated with heparin (1 U/ml) in the absence or presence of a 10-fold molar excess of bi-paratopic sdAbs recognizing antithrombin. Plotted is residual thrombin activity (expressed as ΔOD/min) versus the various types of incubation mixtures. All bi-paratopic sdAbs were able to partially (28-56%) neutralize antithrombin-mediated inhibition of thrombin.

FIG. 7: Effect of bi-paratopic sdAbs on factor Xa activity in the presence of antithrombin and low molecular weight (LMW)-heparin

Residual amidolytic activity of factor Xa towards the synthetic substrate S-2765 was measured in the absence and presence of a 10-fold molar excess antithrombin. Antithrombin was pre-incubated with LMW-heparin (1 U/ml) in the absence or presence of a 10-fold molar excess of bi-paratopic sdAbs recognizing antithrombin. Plotted is residual factor Xa activity (expressed as ΔOD/min) versus the various types of incubation mixtures. All bi-paratopic sdAbs were able to partially (34-68%) neutralize antithrombin-mediated inhibition of factor Xa.

FIG. 8: Effect of bi-paratopic sdAbs on thrombin generation in FVIII-deficient plasma

Presented are examples of thrombin generation curves obtained from FVIII-deficient plasma supplemented or not with various concentrations of FVIII (2.5%, 10% or 100%) or a single dose of bi-paratopic sdAb (10 micromolar). Most efficient among the sdAbs in promoting thrombin generation was KB-AT-002/003.

FIG. 9: Effect of multivalent sdAbs on thrombin activity in the presence of antithrombin and unfractionated heparin

Residual amidolytic activity of thrombin towards the synthetic substrate S-2238 was measured in the absence and presence of a 10-fold molar excess antithrombin. Antithrombin was pre-incubated with heparin (1 U/ml) in the absence or presence of various concentrations of multivalent sdAbs recognizing antithrombin. Plotted is the regained thrombin activity (% thrombin activity in the absence of antithrombin) versus the molar ratio sdAb/antithrombin. Multivalent sdAbs KB-AT-113 and KB-AT-1123 were able to regain >95% of thrombin activity in the presence of antithrombin and heparin.

FIG. 10: Effect of multivalent sdAbs on thrombin generation in FVIII-deficient plasma

Presented are examples of thrombin generation curves obtained from FVIII-deficient plasma supplemented or not with various concentrations of FVIII (2.5%, 10% or 100%) or a single dose of multivalent sdAb (10 micromolar). Most efficient among the sdAbs in promoting thrombin generation were KB-AT-113 and KB-AT-1123.

FIG. 11: Reduced blood loss in FVIII-deficient mice that received KB-AT-002/003

KB-AT-002/003 (10 mg/kg) or vehicle were given to intravenously to FVIII-deficient mice and 10 min after injection, the lateral vein of anesthetized mice was transected at a diameter of 2.3 mm and a depth of 0.7 mm. Blood was collected for a period of 30 min and the volume of shed blood was determined. Blood loss was significantly reduced in mice receiving KB-AT-002/003 compared to control mice receiving vehicle.

FIG. 12: Expression of KB-AT-003/C4BP induces allows arrest of bleeding upon heparin overdosing

The ability of KB-AT-003/C4BP to reduce the bleeding time upon heparin overdose was tested via transient expression of the plasmid pLIVE-KB-AT-003/C4BP in wild-type C57B6/J mice. As a control, mice were given an empty expression plasmid (pLIVE-empty). Four days after gene transfer, mice were a single subcutaneous injection of unfractionated heparin (2000 U/kg). Fifteen minutes after heparin injection, the terminal tip of the tail was amputated in anesthetized mice. Time to arrest of bleeding was monitored and is presented for each mouse. The bleeding time was significantly shorter in mice expressing KB-AT-003/C4BP.

FIG. 13: Reduced blood loss in FVIII-deficient mice that received KB-AT-113.

KB-AT-113 (10 mg/kg) or vehicle were given to intravenously to FVIII-deficient mice and 10 min after injection, the lateral vein of anesthetized mice was transected at a diameter of 2.3 mm and a depth of 0.7 mm. Blood was collected for a period of 60 min and the volume of shed blood was determined. Blood loss was significantly reduced in mice receiving KB-AT-113 compared to control mice receiving vehicle.

FIG. 14: Correction of hemostasis in hemophilic mice

WT-FVIII-SQ and FVIII-AT-0203 were expressed in factor VIII-deficient mice via hydrodynamic gene delivery (HGD; 1.5 microgram/mouse). Five days after HGD, the caudal veins of the anesthetized mice were transected. Blood loss was measured over a 30-min period. The volume of shed blood was determined and is presented for each mouse. No significant difference in blood loss between mice expressing WT-FVIII-SQ and FVIII-AT-0203 was observed, indicating that the introduction of KB-AT-0203 in the factor VIII molecule does not impair its function.

FIG. 15: In vivo survival of FVIII-AT-0203

WT-FVIII-SQ and FVIII-AT-0203 were expressed in factor VIII-deficient mice via hydrodynamic gene delivery (HGD; 100 microgram/mouse). Four days after HGD, plasma was collected. Plasma from factor VIII-deficient mice expressing WT-FVIII-SQ or FVIII-AT-0203 was then infused in factor VIII-deficient mice at a dose of 1 U/mouse. As control, recombinant WT-FVIII-SQ was used at a similar dose. At indicated time-points, blood was collected and factor VIII activity was determined. Residual activity relative the amount injected is plotted against time after injection. FVIII-AT-0203 is removed from the circulation 2.5-fold slower than WT-FVIII-SQ. Symbols: open squares represent mice infused with plasma containing WT-FVIII-SQ; black circles represent mice that received purified recombinant WT-FVIII-SQ; grey squares represent mice infused with plasma containing FVIII-AT-0203.

FIG. 16: Effect of KB-AT-443 on thrombin generation in FVIII-deficient plasma

Presented are examples of thrombin generation curves obtained from FVIII-deficient plasma supplemented or not with various concentrations of FVIII (10% or 100%) or a single dose of KB-AT-443 (4 micromolar). KB-AT-443 strongly enhances thrombin generation in the absence of FVIII.

### EXAMPLES

### Example 1: Binding of Anti-Antithrombin sdAbs to Human and Murine Antithrombin

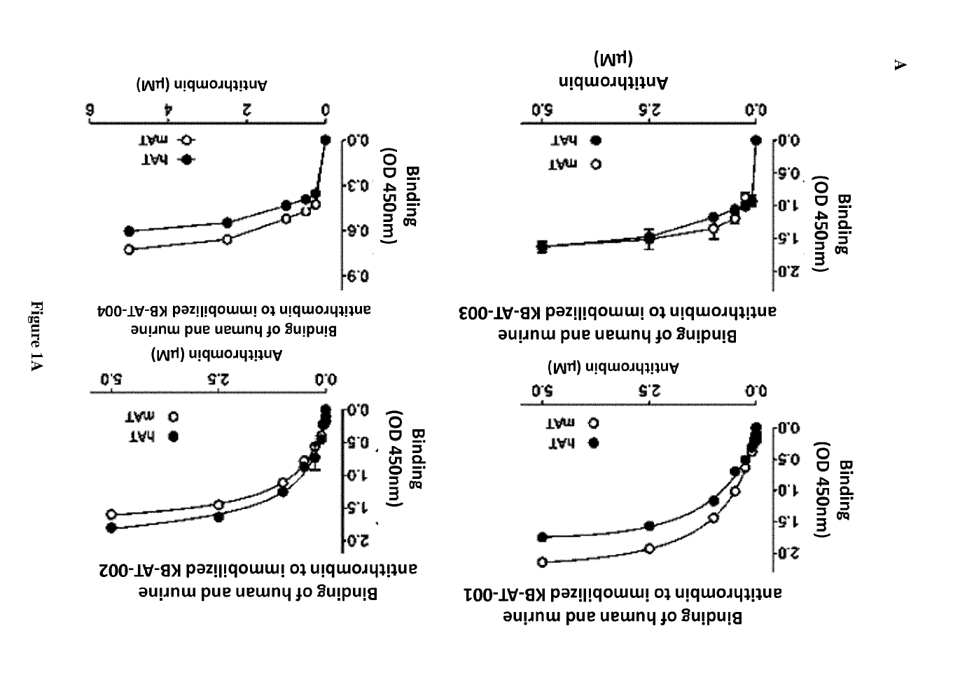

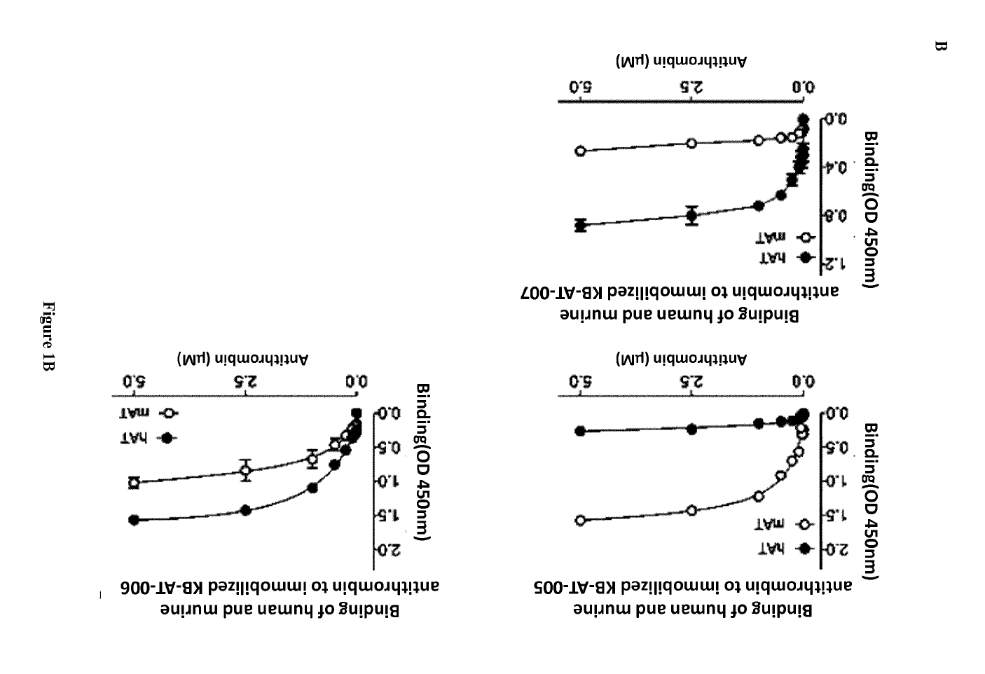

sdAbs recognizing antithrombin (KB-AT-001, -002, -003, -004, -005, -006, and -007) were immobilized (5 microgram/ml) in 10 mM NaHCO₃, 50 mM Na2CO3 (pH 9.5) in a volume of 50 microliter in half-well microtiter plates (Greiner Bio-One, Les Ulis, France) for 16 h at 4° C. After washing the wells three times with 100 microliter/well using Tris-buffered saline (pH 7.6) supplemented with 0.1% Tween-20 (TBS-T), wells were blocked with 100 microliter/well of TBS-T supplemented with 5% skimmed milk for 30 min at 37° C. Wells were washed as described above, and subsequently different concentrations of purified human antithrombin or murine antithrombin (0-5 micromolar diluted in Tris-buffered saline (pH 7.6) supplemented with 5% skimmed milk; 50 microliter/well) were added to each of the immobilized sdAbs and incubated for 2 hours. Wells were then washed three times with 100 microliter/well using TBS-T. Bound antithrombin was probed with peroxidase-labeled polyclonal rabbit anti-antithrombin antibodies (Diagnostica Stago, Asnièrs-sur-Seine, France; Dilution 1/100) for 1 hour at 37° C. with 50 microliter per well. Wells were then washed three times with 100 microliter/well using TBS-T. Residual peroxidase activity was detected by measuring peroxidase-mediated hydrolysis of 3,3′,5,5′-tetramethylbenzidine. OD-values were plotted against antithrombin concentrations (FIG. 1).

sdAbs were considered to recognize human and murine antithrombin similarly, if the difference in max OD-value was less then 30%. Using this criterion, sdAbs KB-AT-001, -002, -003, and -004 displayed similar binding to human and murine antithrombin, sdAbs KB-AT-006 and KB-AT-007 bound more efficiently to human antithrombin compared to murine antithrombin, whereas KB-AT-005 was considerably more efficient in binding to murine antithrombin compared to human antithrombin.

The interaction between sdAbs KB-AT-001, KB-AT-002, KB-AT-003 and KB-AT-006 versus human antithrombin was further analyzed via biolayer-interferometry analysis using Octet-QK equipment in order to determine apparent dissociation constants (KD,app). To this end, sdAbs KB-AT-001, KB-AT-002, KB-AT-003 and KB-AT-006 were diluted in 0.1 M Mes (pH 5.0) to a concentration of 200 microgram/ml for coupling to EDC/NHS-activated amine-reactive biosensors (Fort6bio, Menlo Park, Calif., USA). Sensors were rehydrated in 0.2 ml 0.1 M MES, pH 5.0 for 300 sec. Sensors were then activated via incubation with 0.1 ml 0.2 M EDC/0.095 M NHS mixture for 300 sec and subsequently incubated with 0.1 ml sdAb-solution for 600 sec. Unoccupied amine-reactive sites were quenched by incubating with IM ethanolamine for 180 sec, and sensors were allowed to reach stable baseline levels via incubation with phosphate-buffered saline supplemented with 0.1% Tween-20 (PBS-T) for 300 sec. sdAb-coated sensors were then transferred to wells containing various concentrations of purified antithrombin (0, 12.5, 25 and 50 microgram/ml in PBS-T) and incubated for 600 sec in order to visualize association of antithrombin to immobilized sdAbs. Following this association phase, sensors were transferred to wells containing PBS-T and incubated for 900 sec, allowing dissociation of the antithrombin-sdAb complex. Obtained data were subsequently analyzed using Octet-QK data analysis software (Origin vs 4) to estimate KD,app. This analysis revealed the following values: KD,app=14 nM, 4 nM, 0.4 nM and 22 nM for KB-AT-001, KB-AT-002, KB-AT-003 and KB-AT-006, respectively.

### Example 2: Fusion of Proteins to Anti-Antithrombin sdAbs to Prolong the Half-Life of these Proteins

A construct was established encoding the human von Willebrand factor (VWF)-A1 domain containing a K to A mutation at position 1362 (numbering corresponding to full-length VWF) fused to the bi-paratopic sdAb variant KB-AT-002/003, which combines the sdAbs KB-AT-002 and KB-AT-003 (SEQ ID #38). The mutation was introduced to ensure that the isolated A1 domain would not interact with the platelet receptor glycoprotein Ib□. The resulting protein was designated as VWF-A1/KB-AT-002/003. As a control, VWF-A1/p.K1362A was fused to a non-specific sdAb, which does not react with murine plasma proteins (VWF-A1/KB-UT-01). Purified VWF-A1/KB-AT-002/003 or VWF-A1/KB-UT-01 were given intravenously (10 mg/kg) to wild-type C57B/6 mice. At different time-points after injection (5 min, 15 min, 30 min, 1 h, 3 h, 6 h and 24 h) blood samples were obtained via retro-orbital puncture from isoflurane-anesthetized mice and plasma was prepared by centrifugation (1500 g for 20 min at 22° C.). Residual plasma concentrations were measured using an in-house ELISA that specifically measures human VWF A1 domain, employing murine monoclonal antibody mAb712 as capturing antibody and peroxidase-labeled murine monoclonal antibody mAb724 as probing antibody.

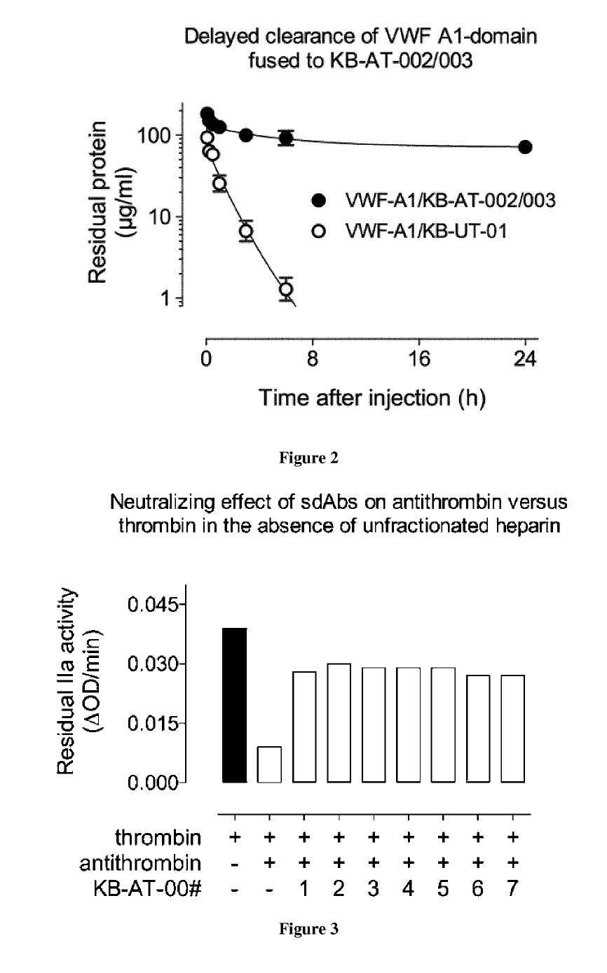

Recovery of at 5 min after injection was significantly higher for VWF-A1/KB-AT-002/003 compared to VWF-A1/KB-UT-01 (92.7±17.7% versus 47.1±6.7%; p=0.012 in unpaired t test with equal SD). We then plotted residual protein concentrations versus time after injection (FIG. 2), revealing a marked difference in residual protein levels at all timepoints after injection. Both proteins appeared to be eliminated from the circulation in a bi-exponential manner. A model for bi-exponential decay was used to calculate the apparent initial and terminal half-lives. The initial half-lives (T½α were calculated to be 0.30 h (95% confidence interval (CI) 0.20-0.60 h) and 0.03 h (95% CI 0.02-0.05 h) for VWF-A1/KB-AT-002/003 and VWF-A1/KB-UT-01, respectively. The terminal half-lives (T½β were calculated to be 38 h (95% CI 21-178 h) and 0.7 h (95% CI 0.5-1.0 h) for VWF-A1/KB-AT-002/003 and VWF-A1/KB-UT-01, respectively. This demonstrates that fusion of a protein with a relatively short half-life to sdAbs recognizing antithrombin considerably increases the circulatory half-life of such protein.

### Example 3: Neutralization of Antithrombin-Mediated Inhibition of Thrombin in the Absence of Heparin

Purified human antithrombin (5 nM) was incubated in the absence or presence of monovalent sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to thrombin (0.5 nM) in the presence of the amidolytic substrate S-2238 and hydrolysis was monitored for 20 min by measuring optical density (OD) at wavelength 405 nm. Plotted in FIG. 3 is the velocity of substrate hydrolysis (delta OD/min) for thrombin in the absence or presence of antithrombin as well as for the mixtures containing thrombin, antithrombin and sdAbs. These data show that each of the sdAbs against antithrombin are able to increase thrombin activity in the presence of antithrombin, which is compatible with the sdAbs interfering with the inhibitory activity of antithrombin towards thrombin under these conditions. The percentage by which the sdAbs neutralize antithrombin-mediated thrombin inhibition is summarized in table 1.

TABLE 1
neutralization of antithrombin by monovalent
sdAbs in the absence of heparin:

| sdAb | % antithrombin neutralization |
|---|---|
| KB-AT-001 | 62 |
| KB-AT-002 | 65 |
| KB-AT-003 | 67 |
| KB-AT-004 | 55 |
| KB-AT-005 | 57 |
| KB-AT-006 | 58 |
| KB-AT-007 | 61 |

Thus, all monovalent sdAbs tested were able to partially neutralize antithrombin activity towards thrombin (FIG. 3 and Table 1). However, none of them was able to fully block antithrombin activity.

### Example 4: Lack of Neutralization of Antithrombin-Mediated Inhibition of Thrombin by Monovalent sdAbs in the Presence of Heparin

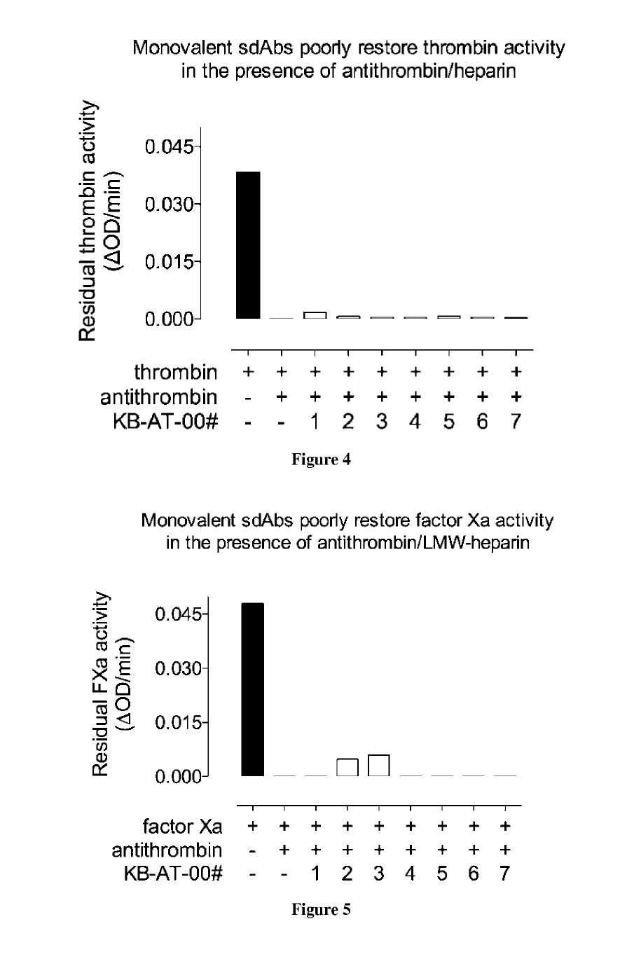

Purified human antithrombin (5 nM) was incubated with unfractionated heparin (1 U/ml) in the absence or presence of monovalent sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to thrombin (0.5 nM) in the presence of the amidolytic substrate S-2238 and hydrolysis was monitored for 20 min by measuring optical density (OD) at wavelength 405 nm. Plotted in FIG. 4 is the velocity of substrate hydrolysis (delta OD/min) for thrombin in the absence or presence of antithrombin & heparin as well as for the mixtures containing thrombin, antithrombin, heparin and sdAbs. These data show that none of the sdAbs against antithrombin are able to increase thrombin activity in the presence of antithrombin & heparin. The percentage by which the sdAbs neutralize antithrombin-mediated thrombin inhibition was less than 5%. This demonstrates that monovalent sdAbs lack the capacity to interfere with antithrombin-mediated inhibition of thrombin in the presence of unfractionated heparin.

### Example 5: Lack of Neutralization of Antithrombin-Mediated Inhibition of Factor Xa by Monovalent sdAbs in the Presence of Heparin

Purified human antithrombin (5 nM) was incubated with low molecular weight (LMW-heparin; Lovenox; 1 U/ml) in the absence or presence of monovalent sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to factor Xa (0.5 nM) in the presence of the amidolytic substrate S-2765 and hydrolysis was monitored for 20 min by measuring optical density (OD) at wavelength 405 nm. Plotted in FIG. 5 is the velocity of substrate hydrolysis (delta OD/min) for factor Xa in the absence or presence of antithrombin & LMW-heparin as well as for the mixtures containing factor Xa, antithrombin, heparin and sdAbs. These data show that none of the sdAbs against antithrombin are able to substantially increase factor Xa activity in the presence of antithrombin & LMW-heparin. The percentage by which the sdAbs neutralize antithrombin-mediated factor Xa inhibition was less than 15%. This demonstrates that monovalent sdAbs poorly interfere with antithrombin-mediated inhibition of factor Xa in the presence of LMW-heparin.

### Example 6: Neutralization of Antithrombin-Mediated Inhibition of Thrombin by Bi-Paratopic sdAbs in the Presence of Heparin

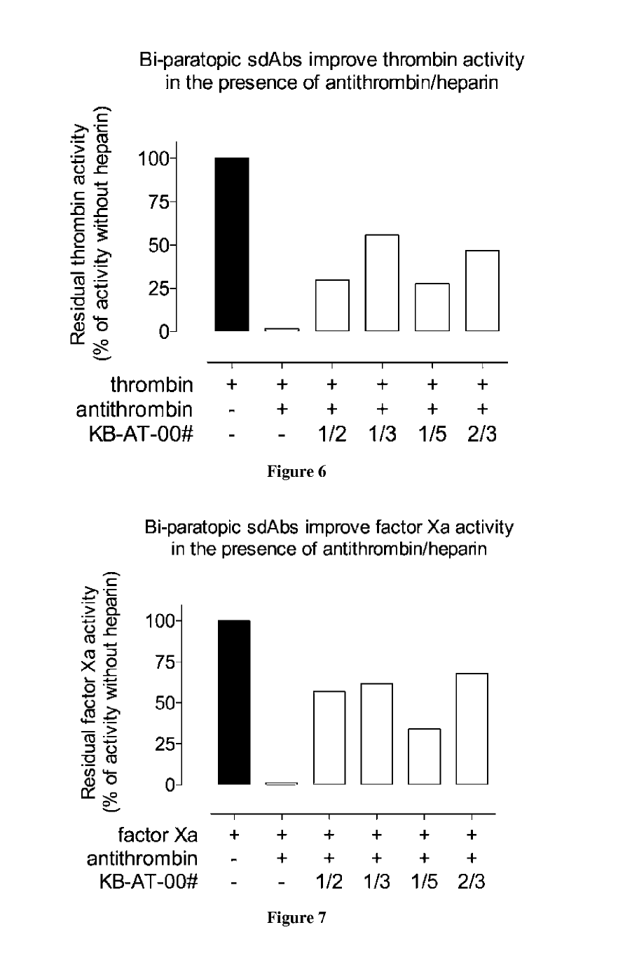

Constructs were established encoding sdAb combinations consisting of two different sdAbs against antithrombin (bi-paratopic sdAbs): KB-AT-001/002 (SEQ ID#30), KB-AT-001/003 (SEQ ID#31), KB-AT-001/005 (SEQ ID#32) and KB-AT-002/003 (SEQ ID#29). Purified human antithrombin (5 nM) was incubated with unfractionated heparin (1 U/ml) in the absence or presence of bi-paratopic sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to thrombin (0.5 nM) in the presence of the amidolytic substrate S-2238 and hydrolysis was monitored for 20 min by measuring optical density (OD) at wavelength 405 nm. Plotted in FIG. 6 is the percentage of residual thrombin activity, compared to thrombin activity in the absence antithrombin & heparin. Whereas residual thrombin activity in the presence of antithrombin & heparin alone was less than 5%, significantly higher thrombin activity was measured in the presence of the bi-paratopic sdAbs. The percentage by which the bi-paratopic sdAbs neutralize antithrombin-mediated thrombin inhibition is summarized in table 2. These data demonstrate that combining different sdAbs renders these combinations with the ability to neutralize antithrombin function in the presence of unfractionated heparin.

TABLE 2
neutralization of antithrombin by bi-paratopic
sdAbs in the presence of heparin:

| sdAb combination | % antithrombin neutralization |
|---|---|
| KB-AT-001/002 | 30 |
| KB-AT-001/003 | 56 |
| KB-AT-001/005 | 28 |
| KB-AT-002/003 | 47 |

### Example 7: Neutralization of Antithrombin-Mediated Inhibition of Factor Xa in the Presence of Heparin by Bi-Paratopic sdAbs

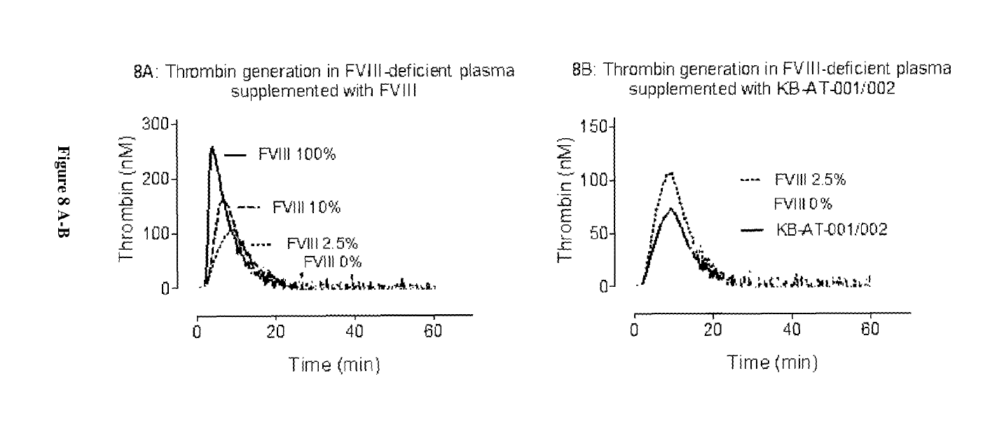

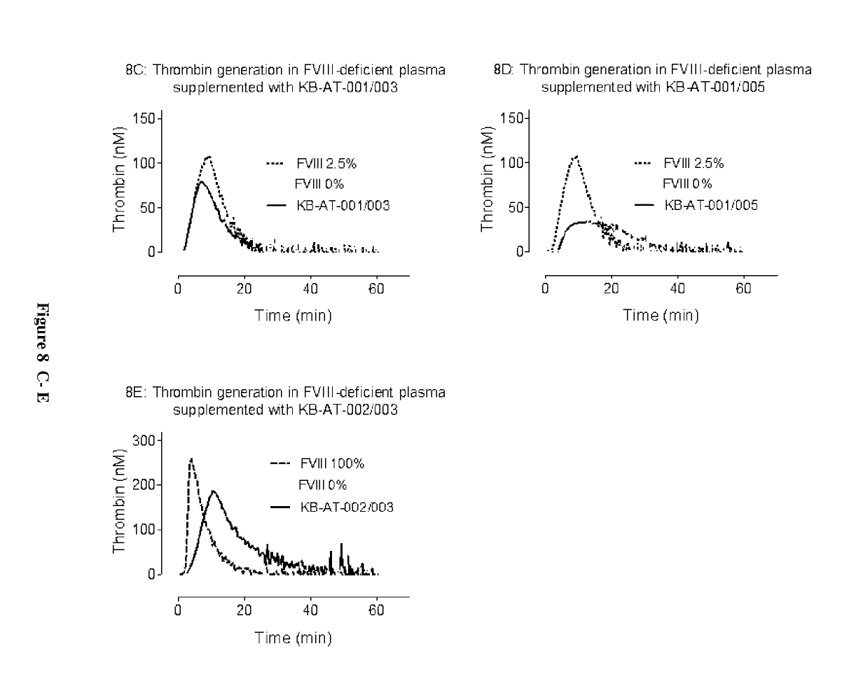

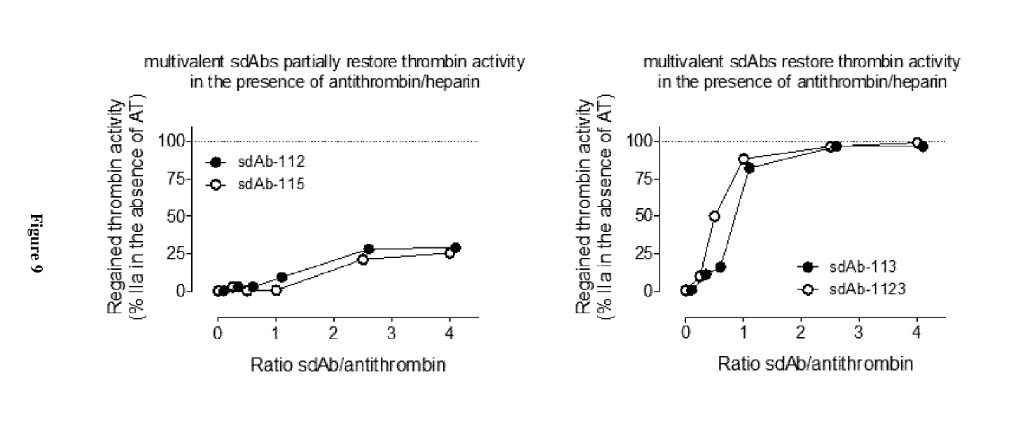

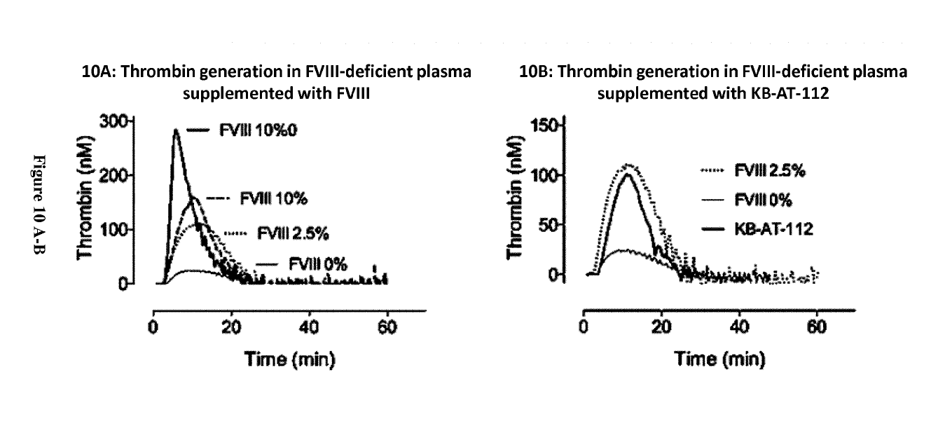

Bi-paratopic sdAbs were also tested for their capacity to neutralize antithrombin activity in the presence of LMW-heparin towards factor Xa. Purified human antithrombin (5 nM) was incubated with low molecular weight (LMW-heparin; Lovenox; 1 U/ml) in the absence or presence of bi-paratopic sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to factor Xa (0.5 nM) in the presence of the amidolytic substrate S-2765 and hydrolysis was monitored for 20 min by measuring optical density (OD) at wavelength 405 nm. Plotted in FIG. 7 is the percentage of residual factor Xa activity, compared to factor Xa activity in the absence antithrombin & LMW-heparin. Whereas residual factor Xa activity in the presence of antithrombin & LMW-heparin alone was less than 5%, significantly higher factor Xa activity was measured in the presence of the bi-paratopic sdAbs. The percentage by which the bi-paratopic sdAbs neutralize antithrombin-mediated thrombin inhibition is summarized in table 3. These data demonstrate that combining different sdAbs renders these combinations with the ability to neutralize antithrombin function in the presence of LMW-heparin

TABLE 3
neutralization of antithrombin by bi-paratopic
sdAbs in the presence of LMW-heparin:

| sdAb | % antithrombin neutralization |
|---|---|
| KB-AT-001/002 | 57 |
| KB-AT-001/003 | 62 |
| KB-AT-001/005 | 34 |
| KB-AT-002/003 | 68 |

### Example 8: Effect of Bi-Paratopic sdAbs in Thrombin Generation Assay Using Hemophilic Plasma

Bi-paratopic sdAbs were analyzed for their capacity to restore thrombin generation in factor VIII (FVIII)-deficient plasma. Thrombin generation was measured according to the method described by Hemker et al (pathophysiology of haemostasis and thrombosis (2002) 32:249-253), in a Fluoroscan Ascent fluorometer (Thermolabsystems OY, Helsink, Finland) equipped with a dispenser. Briefly, 80 μl of plasma supplemented with either saline (control), purified FVIII (0.025, 0.1, and 1 U/ml Kogenate® FS, Bayer HealthCare, Puteaux, France) or with bi-paratopic sdAb (10 micromolar) were dispensed into round-bottom 96-well microtiter plates. Twenty microliter of a mixture containing TF (recombinant lipidated human tissue factor, Innovin®, obtained from Dade Behring) and phospholipids (PL) vesicles was added to the plasma sample to obtain a final concentration of 1 pM TF and 4 micromolar PL vesicles. PL vesicles were prepared from L-α-Phosphatidyl-L-serine (PS) L-α-phosphatidylethanolamine (PE) and L-α-phosphatidylcholine (PC) (Avanti Polarlipids, Alabaster, Ala., USA) to a ratio of PC:PE:PS=3:1:1 and were of nominal 100 nm-diameter.

Thrombin generation was triggered by adding 20 microliter of starting reagent containing fluorogenic substrate and CaCl2. Fluorogenic substrate I-1140 (Z-Gly-Gly-Arg-AMC) was from Bachem AG (Bubendorf, Switzerland). Kinetics of thrombin generation in clotting plasma was monitored for 60 min at 37° C. using a calibrated automated thrombogram and analyzed using the Thrombinoscope-software (Thrombinoscope B.V., Maastricht, the Netherlands). Four wells were needed for each experiment, two wells to measure thrombin generation of a plasma sample and two wells for calibration. All experiments were carried out in triplicate and the mean value was reported. Endogenous thrombin potential (ETP), i.e. area under the curve, peak thrombin and lag time for thrombin detection were determined.

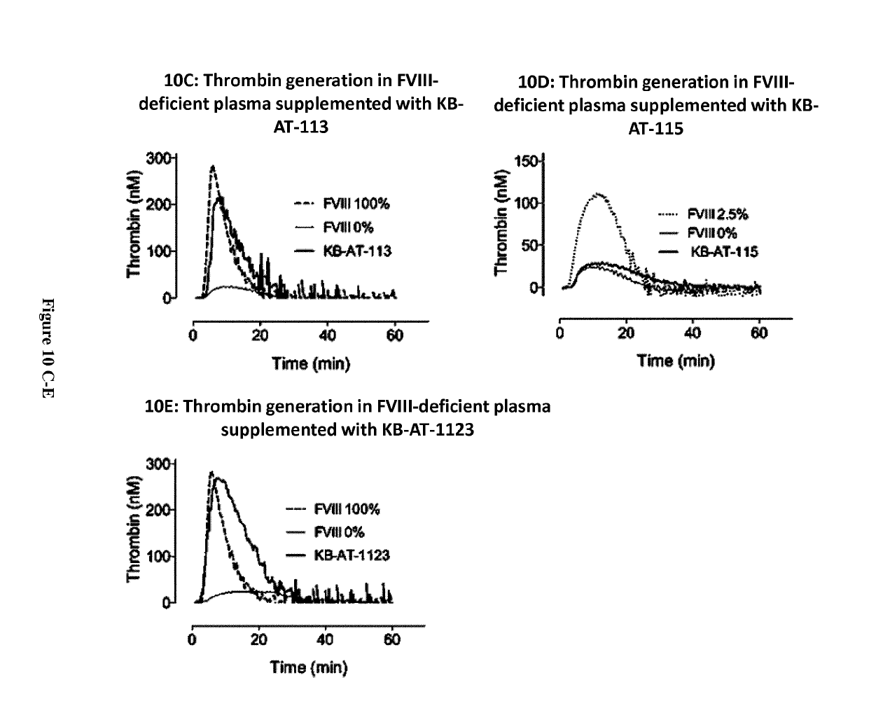

In FIG. 8, examples of thrombin generation curves are represented. In FIG. 8A, thrombin generation of FVIII-deficient plasma and FVIII-deficient plasma spiked with different concentrations of FVIII (2.5%, 10% and 100%) is shown. In FIG. 8B, thrombin generation of FVIII-deficient plasma, FVIII-deficient plasma spiked with 2.5% FVIII and FVIII-deficient plasma spiked with KB-AT-001/002 (10 micromolar) is shown. In FIG. 8C, thrombin generation of FVIII-deficient plasma, FVIII-deficient plasma spiked with 2.5% FVIII and FVIII-deficient plasma spiked with KB-AT-001/003 (10 micromolar) is shown. In FIG. 8D, thrombin generation of FVIII-deficient plasma, FVIII-deficient plasma spiked with 2.5% FVIII and FVIII-deficient plasma spiked with KB-AT-001/005 (10 micromolar) is shown. In FIG. 8E, thrombin generation of FVIII-deficient plasma, FVIII-deficient plasma spiked with 2.5% FVIII and FVIII-deficient plasma spiked with KB-AT-002/003 (10 micromolar) is shown. The thrombin-generation parameters are summarized in Table 4. These thrombin generation curves show that sdAb combination KB-AT-001/002, KB-AT-001/003 and KB-AT-001/005 stimulate thrombin generation in FVIII-deficient plasma only to a limited extent, reaching thrombin generation levels that correspond to less than 2.5% of FVIII. In contrast, the combination KB-AT-002/003 is much more efficient in the amount of thrombin generated (1.7 fold more compared to 100% FVIII). Nevertheless, the lag-time before thrombin generation is initiated is still significantly delayed compared to the presence of 100% FVIII (see Table 4).

TABLE 4
thrombin generation parameters for bi-paratopic sdAbs:

| | Lag-time | ETP | Thrombin Peak |
|---|---|---|---|
| | (min) | (nM) | (nM) |
| FVIII 0% | 6.3 | 502 | 39 |
| FVIII 2.5% | 2.7 | 1121 | 107 |
| FVIII 10% | 3.0 | 1418 | 170 |
| FVIII 100% | 2.3 | 1618 | 236 |
| KB-AT-001/002 | 2.7 | 977 | 81 |
| KB-AT-001/003 | 2.3 | 1008 | 83 |
| KB-AT-001/005 | 5.3 | 609 | 33 |
| KB-AT-002/003 | 3.3 | 2680 | 185 |

### Example 9: Neutralization of Antithrombin-Mediated Inhibition of Thrombin in the Presence of Heparin by Multivalent sdAbs

Constructs were established encoding sdAb combinations consisting of two or three different sdAbs against antithrombin, in which at least one of the sdAbs was present in duplicate: (multivalent sdAbs): KB-AT-001/001/002 (SEQ ID#33, referred to as KB-AT-112), KB-AT-001/001/003 (SEQ ID#34; KB-AT-113), KB-AT-001/001/005 (SEQ ID#35; KB-AT-115) and KB-AT-001/001/002/003 (SEQ ID#36; KB-AT-1123).

Purified human antithrombin (5 nM) was incubated with unfractionated heparin (1 U/ml) in the absence or presence of multivalent sdAbs (100 nM) for 15 min in TBSC-buffer (Tris-buffered saline supplemented with 50 mM CaCl2, 0.1% protease-free bovine serum albumin, 0.1% PEG8000, pH 7.4) at 37° C. This mixture was subsequently added to thrombin (0.5 nM) in the presence of the amidolytic substrate S-2238 and hydrolysis was monitored for 20 min by measuring optical density (OD) at wavelength 405 nm. Plotted in FIG. 9 is the percentage of residual thrombin activity, compared to thrombin activity in the absence antithrombin & heparin. Whereas residual thrombin activity in the presence of antithrombin & heparin alone was less than 5%, significantly higher thrombin activity was measured in the presence of the multivalent sdAbs. The percentage by which the multivalent sdAbs neutralize antithrombin-mediated thrombin inhibition is summarized in table 5. These data demonstrate that combining different sdAbs renders these combinations with the ability to neutralize antithrombin function in the presence of unfractionated heparin.

TABLE 5
neutralization of antithrombin by multivalent
sdAbs in the presence of heparin

| sdAb combination | % antithrombin neutralization |
|---|---|
| KB-AT-112 | 29 |
| KB-AT-113 | 97 |
| KB-AT-115 | 26 |
| KB-AT-1123 | 99 |

### Example 10: Effect of Multivalent sdAbs in Thrombin Generation Assay Using Hemophilic Plasma

Multivalent sdAbs were analyzed for their capacity to restore thrombin generation in factor VIII (FVIII)-deficient plasma. Thrombin generation was measured according to the method described by Hemker et al (pathophysiology of haemostasis and thrombosis (2002) 32:249-253), in a Fluoroscan Ascent fluorometer (Thermolabsystems OY, Helsink, Finland) equipped with a dispenser. Briefly, 80 μl of plasma supplemented with either saline (control), purified FVIII (0.025, 0.1, and 1 U/ml Kogenate® FS, Bayer HealthCare, Puteaux, France) or with multivalent sdAb (10 micromolar) were dispensed into round-bottom 96-well microtiter plates. Twenty microliter of a mixture containing TF (recombinant lipidated human tissue factor, Innovin®, obtained from Dade Behring) and phospholipids (PL) vesicles was added to the plasma sample to obtain a final concentration of 1 pM TF and 4 micromolar PL vesicles. PL vesicles were prepared from L-α-Phosphatidyl-L-serine (PS) L-α-phosphatidylethanolamine (PE) and L-α-phosphatidylcholine (PC) (Avanti Polarlipids, Alabaster, Ala., USA) to a ratio of PC:PE:PS=3:1:1 and were of nominal 100 nm-diameter.

Thrombin generation was triggered by adding 20 microliter of starting reagent containing fluorogenic substrate and CaCl2. Fluorogenic substrate I-1140 (Z-Gly-Gly-Arg-AMC) was from Bachem AG (Bubendorf, Switzerland). Kinetics of thrombin generation in clotting plasma was monitored for 60 min at 37° C. using a calibrated automated thrombogram and analyzed using the Thrombinoscope-software (Thrombinoscope B.V., Maastricht, the Netherlands). Four wells were needed for each experiment, two wells to measure thrombin generation of a plasma sample and two wells for calibration. All experiments were carried out in triplicate and the mean value was reported. Endogenous thrombin potential (ETP), i.e. area under the curve, peak thrombin, lag time for thrombin detection and time to thrombin peak were determined.

In FIG. 10, examples of thrombin generation curves are represented. In FIG. 10A, thrombin generation of FVIII-deficient plasma and FVIII-deficient plasma spiked with different concentrations of FVIII (2.5%, 10% and 100%) is shown. In FIG. 10B, thrombin generation of FVIII-deficient plasma, FVIII-deficient plasma spiked with 2.5% FVIII and FVIII-deficient plasma spiked with KB-AT-112 (10 micromolar) is shown. In FIG. 10C, thrombin generation of FVIII-deficient plasma, FVIII-deficient plasma spiked with 100% FVIII and FVIII-deficient plasma spiked with KB-AT-113 (10 micromolar) is shown. In FIG. 10D, thrombin generation of FVIII-deficient plasma, FVIII-deficient plasma spiked with 2.5% FVIII and FVIII-deficient plasma spiked with KB-AT-115 (10 micromolar) is shown. In FIG. 10E, thrombin generation of FVIII-deficient plasma, FVIII-deficient plasma spiked with 100% FVIII and FVIII-deficient plasma spiked with KB-AT-1123 (10 micromolar) is shown. The thrombin-generation parameters are summarized in Table 6. These thrombin generation curves show that sdAb combination KB-AT-112 and KB-AT-115 stimulate thrombin generation in FVIII-deficient plasma only to a limited extent, reaching thrombin generation levels that correspond to less than 2.5% of FVIII. Combination KB-AT-113 is more efficient, as its presence results in more thrombin generation compared to the presence of 10% FVIII. Nevertheless, less thrombin is generated compared to 100% FVIII. In contrast, the combination KB-AT-1123 is much more efficient in the amount of thrombin generated (1.4 fold more compared to 100% FVIII). Moreover, the lag-time to which thrombin generation is initiated is significantly shorter compared to the presence of 100% FVIII (see Table 6). These data show that different combinations of sdAbs all modulate thrombin generation in FVIII-deficient plasma to a different extent, and that only certain combinations (in this case KB-AT-1123) are able to outperform FVIII in this thrombin generation test, by producing more thrombin and by quicker initiating thrombin formation.

TABLE 6
thrombin generation parameters for multivalent sdAbs

| | Lag-time | ETP | Thrombin Peak |
|---|---|---|---|
| | (min) | (nM) | (nM) |
| FVIII 0% | 5.3 | 107 | 24 |
| FVIII 2.5% | 4.0 | 854 | 104 |
| FVIII 10% | 3.7 | 1236 | 155 |
| FVIII 100% | 3.3 | 1814 | 288 |
| KB-AT-112 | 5.8 | 540 | 95 |
| KB-AT-113 | 3.3 | 1428 | 221 |
| KB-AT-115 | 5.3 | 228 | 26 |
| KB-AT-1123 | 2.0 | 2628 | 264 |

### Example 11: Effect of Bi-Paratopic sdAb KB-AT-002/003 on Blood Loss in Tail Vein Transection Assay Using Hemophilic Mice

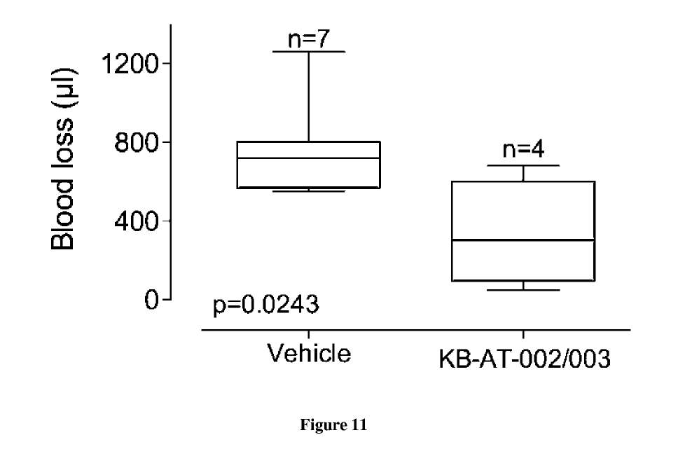

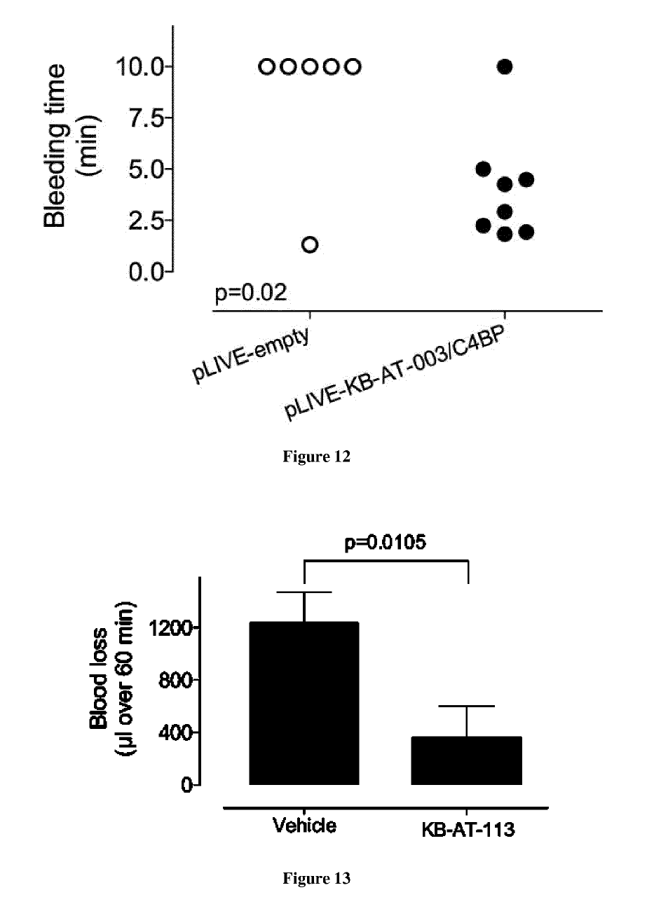

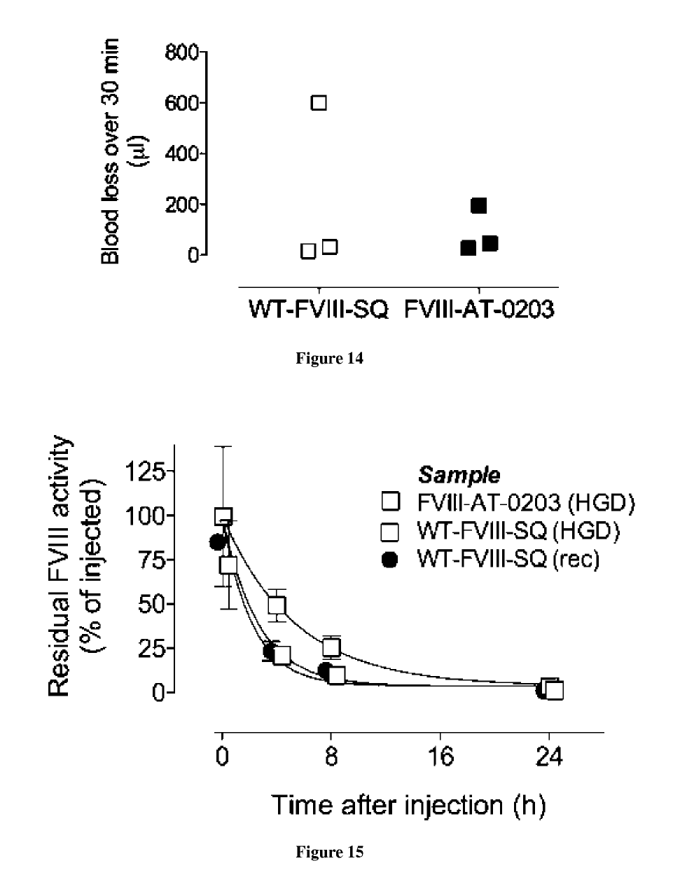

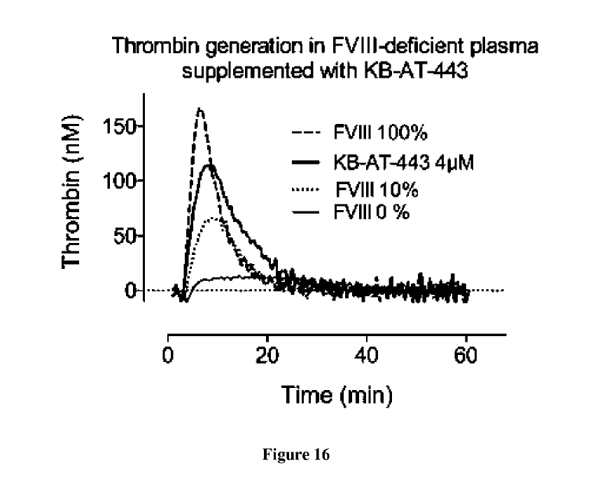

8-12 week old hemophilic mice were given vehicle (saline) or sdAb KB-AT-002/003 (10 mg/kg) via intravenous tail injection. Ten minutes after injection, the lateral vein of isoflurane-anesthetized mice were cut at a depth of 0.7 mm, there where the diameter of the tail was 2.3 mm. The transected tail was immersed immediately after transection in a 10 ml tube full of warm physiological saline. Blood was collected for 30 min at 37° C. After 30 min, the mixture of blood and physiological saline was centrifuged at 1500 g. The red blood cells pellet was then lysed in H2O and the amount of hemoglobin was obtained by reading the absorbance at 416 nm. The volume of blood lost in each sample was calculated from a standard curve, which is obtained by lysing defined volumes (20 microliter, 40 microliter, 60 microliter, 80 microliter and 100 microliter) of mouse blood in H2O to extract hemoglobin as described above. Blood loss for vehicle- and KB-AT-002/003 treated mice is presented in FIG. 11. Infusion of KB-AT-002/003 results in reduced blood loss in hemophilic mice. Blood loss was 334±133 microliter (mean±SE; n=4) for mice receiving KB-AT-002/003 (10 mg/kg) and 755±90 microliter (n=7) for vehicle-receiving mice. This difference was statistically significant (p=0.0243) when analyzed in an unpaired t-test with equal SD. These data show that neutralization of antithrombin by KB-AT-002/003 restores haemostasis, at least partially, in hemophilic mice.

### Example 12: In Vivo Expression of a Heptameric sdAb-C4BP Fusion Protein Neutralizes Bleeding Tendency Induced by Heparin Overdosing

A construct was established encoding KB-AT-003 fused to a 57-amino acid peptide motif of C4BP, which allows heptamerization of the protein (SEQ ID#40; referred to as KB-AT-003/C4BP). The cDNA encoding KB-AT-003/C4BP was cloned into the pLIVE-plasmid (Mirus Bio, Madison, Wis., USA). Empty pLIVE-plasmids were used as negative control. Plasmids (100 microgram/mouse) were injected into wild-type C57B6 mice via hydrodynamic gene transfer: plasmids are diluted in 0.9% saline with the volume corresponding to 10% of the animal's bodyweight (i.e. 2 ml for a 20-gram mouse). The solution is injected in the tail vein within 5 seconds. Four days after gene transfer, mice were given an single subcutaneous injection of unfractionated heparin (2000 U/kg), a dose sufficient to induce bleeding. Fifteen minutes after injection of heparin, the terminal 3 mm of the tail-tip was amputated from ketamine/xylazine-anesthetized mice. The amputated tail was immersed immediately after transection in a 50 ml tube full of physiological saline (37° C.) and the time to the arrest of bleeding was monitored during a 10-min observation period. The time to bleeding arrest for each mouse is presented in FIG. 14. Bleeding time was significantly longer in mice given the control empty pLIVE-plasmid compared to mice that had received the plasmid encoding KB-AT-003/C4BP (8.6±3.6 min versus 4.1±2.7 min for pLIVE-empty and pLIVE-KB-AT-003/C4BP, respectively; mean±SD; p=0.02 in unpaired t-test with equal SD). Indeed, bleeding did not stop during the 10-min observation period in 5 of 6 control mice, whereas 7 of 8 mice that expressed KB-AT-003/C4BP stopped bleeding in 5 min or less.

### Example 13: Binding of sdAb to Antithrombin Present in Plasma of Different Species

sdAbs KB-AT-001, -002. -003, -004, -005, -006, and -007 were immobilized (10 microgram/ml) in 10 mM NaHCO₃, 50 mM Na2CO3 (pH 9.5) in a volume of 50 microliter in half-well microtiter plates (Greiner Bio-One, Les Ulis, France) for 16 h at 4° C. As a positive control, polyclonal anti-antithrombin antibodies (MATIII-EIA kit, Affinity biologicals, Ancaster Canada) were immobilized in a similar fashion. As a negative control, the anti-von Willebrand sdAb KB-VWF-006 was immobilized. After washing the wells three times with 100 microliter/well using Tris-buffered saline (pH 7.6) supplemented with 0.1% Tween-20 (TBS-T), wells were blocked with 100 microliter/well of TBS-T supplemented with 3% bovine serum albumin (BSA) for 30 min at 37° C. Wells were washed as described above, and subsequently the following plasma preparations (diluted ¼ in TBS-T/3% BSA, 100 microliter per well, 2 hours at 37° C.) were added to each of the immobilized sdAbs and both types of control wells: rabbit plasma, canine plasma, simian plasma, bovine plasma, porcine plasma, rat plasma, murine plasma and human plasma.

Wells were then washed three times with 100 microliter/well using TBS-T. Bound antithrombin was probed with peroxidase-labeled polyclonal anti-antithrombin antibodies (MATIII-EIA kit, Affinity biologicals, Ancaster Canada; diluted 1/100) for 2 hours at 37° C. with 50 microliter per well. Wells were then washed three times with 100 microliter/well using TBS-T. Residual peroxidase activity was detected by measuring peroxidase-mediated hydrolysis of 3,3′,5,5′-tetramethylbenzidine.

Negative binding (−) was defined as optical density (OD) being ≤0.1, moderate positive binding (+) was defined as OD being >0.1 and <0.5, strongly positive binding (++) was defined as OD being ≥0.5. Based on these definitions, none of the sdAbs displayed moderate or strongly positive binding to the negative control (Table 1). All plasma preparations had moderate or strongly positive binding to the positive control (polyclonal anti-antithrombin antibodies). The binding of the plasma preparations to the different sdAbs is summarized in Table 7.

Table 7 belonging to example 16: Binding of sdAbs to antithrombin of different species

| | Rab- | Ca- | Sim- | Bo- | Por- | | | |
|---|---|---|---|---|---|---|---|---|
| | bit | nine | ian | vine | cine | Rat | Mouse | Human |
| KB-AT-001 | + | − | ++ | − | − | + | ++ | + |
| KB-AT-002 | ++ | ++ | ++ | ++ | ++ | ++ | ++ | ++ |
| KB-AT-003 | − | + | ++ | − | − | + | ++ | ++ |
| KB-AT-004 | − | + | + | − | + | + | ++ | + |
| KB-AT-005 | − | − | + | − | − | − | + | + |
| KB-AT-006 | ++ | + | ++ | − | + | + | + | + |
| KB-AT-007 | − | − | ++ | − | − | − | + | + |
| Positive ctl | + | ++ | ++ | + | ++ | ++ | ++ | ++ |
| Negative ctl | − | − | − | − | − | − | − | − |

Positive ctl: positive control, polyclonal anti-antithrombin antibodies (Affinity Biologicals).
Negative ctl: anti-VWF sdAb KB-VWF-006 immobilized.
−: Negative binding defined as OD being ≤0.1;
+: Moderate positive binding defined as OD being >0.1-<0.5;
++: Strongly positive binding defined as being ≥0.5

### Example 14: Effect of Multivalent sdAb KB-AT-113 on Blood Loss in Tail Vein Transection Assay Using Hemophilic Mice

8-12 week old hemophilic mice were given vehicle (saline) or sdAb KB-AT-113 (10 mg/kg) via intravenous tail injection. Ten minutes after injection, the lateral vein of isoflurane-anesthetized mice were cut at a depth of 0.7 mm, there where the diameter of the tail was 2.3 mm. The transected tail was immersed immediately after transection in a 10 ml tube full of warm physiological saline. Blood was collected for 60 min at 37° C. After 60 min, the mixture of blood and physiological saline was centrifuged at 1500 g. The red blood cells pellet was then lysed in H2O and the amount of hemoglobin was obtained by reading the absorbance at 416 nm. The volume of blood lost in each sample was calculated from a standard curve, which is obtained by lysing defined volumes (20 microliter, 40 microliter, 60 microliter, 80 microliter and 100 microliter) of mouse blood in H2O to extract hemoglobin as described above. Blood loss for vehicle- and KB-AT-113 treated mice is presented in FIG. 13. Infusion of KB-AT-113 results in reduced blood loss in hemophilic mice. Blood loss was 363±238 microliter (mean±SE; n=3) for mice receiving KB-AT-113 (10 mg/kg) and 1235±233 microliter (n=3) for vehicle-receiving mice. This difference was statistically significant (p=0.0105) when analyzed in an unpaired t-test with equal SD. These data show that neutralization of antithrombin by KB-AT-113 restores haemostasis, at least partially, in hemophilic mice.

### Example 15: Expression of FVIII-AT-0203 Fusion Protein Corrects Hemostasis in Hemophilic Mice

cDNA constructs encoding wild-type B-domainless FVIII (WT-FVIII-SQ) and FVIII-AT-0203 were cloned into the pLIVE-plasmid (Mirus Bio, WI, USA). Plasmids (1.5 microgram/mouse) were injected into factor VIII-deficient mice via hydrodynamic gene transfer: plasmids are diluted in 0.9% saline with the volume corresponding to 10% of the animal's weight (i.e. 2 ml for a 20-gram mouse). The solution is injected in the tail vein within 5 seconds. Four days after gene transfer, blood was collected via retro-orbital puncture from isoflurane anesthetized mice and plasma was prepared by centrifugation (1500 g for 20 min at 22° C.). Plasma was then used to measure FVIII activity using a chromogenic two-stage activity assay (Biophen FVIII:C; Hyphen Biomed, Neuville-sur-Oise, France). Average FVIII activity was 0.14±0.07 U/ml for WT-FVIII-SQ (n=3) and 0.12±0.06 U/ml for FVIII-AT-0203. Five days after gene transfer, the lateral tail vein of isoflurane-anesthetized mice was transected at a diameter of 2.3 mm and a depth of 0.7 mm. Blood was collected for a period of 30 min and the volume of blood loss was determined (FIG. 14). Blood loss was 216±333 microliter for mice expressing WT-FVIII-SQ (mean±SD; n=3), while blood loss was 89±92 microliter for mice expressing FVIII-AT-0203 (mean±SD; n=3). Blood loss was not significantly different between both variants. Thus, FVIII-AT-0203 is at least as efficient as WT-FVIII-SQ in correcting the hemostatic deficit in factor VIII-deficient mice.

### Example 16: FVIII-AT-0203 has a Longer Circulatory Survival as WT-FVIII-SQ

cDNA constructs encoding wild-type B-domainless FVIII (WT-FVIII-SQ) and FVIII-AT-0203 were cloned into the pLIVE-plasmid (Mirus Bio, WI, USA). Plasmids (100 microgram/mouse) were injected into factor VIII-deficient mice (n=2 per construct) via hydrodynamic gene transfer: plasmids are diluted in 0.9% saline with the volume corresponding to 10% of the animal's weight (i.e. 2 ml for a 20-gram mouse). The solution is injected in the tail vein within 5 seconds. Four days after gene transfer, blood was collected via retro-orbital puncture from isoflurane anesthetized mice and plasma was prepared by centrifugation (1500 g for 20 min at 22° C.). Plasma was then used to measure FVIII activity using a chromogenic two-stage activity assay (Biophen FVIII:C; Hyphen Biomed, Neuville-sur-Oise, France). FVIII activity was 3.5 and 4.5 U/ml for WT-FVIII-SQ and 5.6 and 4.4 U/ml for FVIII-AT-0203. Plasma from the mice was used for intravenous infusion into the tail vein of factor VIII-deficient mice. Dosing was 1 U factor VIII/mouse. As control, recombinant WT-FVIII-SQ was infused. At indicated time-points, blood was collected via retro-orbital puncture from isoflurane-anesthetized mice and plasma was prepared by centrifugation (1500 g for 20 min at 22° C.). Residual factor VIII activity was measured in a chromogenic two-stage activity assay. Residual activity relative to the amount injected (mean±SD; n=3-4 for each time-point) was plotted against the time after injection (FIG. 15). This approach revealed that at time-points 4 h, 8 h and 24 h after injection, residual factor VIII levels were significantly lower for WT-FVIII-SQ compared to FVIII-AT-0203. Values were as follows:

| Time after | WT-FVIII-SQ | FVIII-AT-0203 | |
|---|---|---|---|
| infusion | Relative activity | Relative activity | |
| (h) | (%) | (%) | p-value |
| 4 | 21.0 ± 5.2 | 49.3 ± 9.2 | 0.005 |
| 8 | 9.7 ± 1.5 | 25.3 ± 6.7 | 0.017 |
| 24 | 1.1 ± 0.5 | 3.5 ± 1.0 | 0.014 |

Statistical analysis between WT-FVIII-SQ was determined using multiple t-test using the Holm-Sidak method (GraphPrism vs 6.0 h for Mac OS X; GraphPad SoftWare). The higher residual levels of FVIII-AT-0203 compared to WT-FVIII-SQ translated in a calculated half-life that was 2.5-fold increased (3.7 h [95% confidence interval 2.4-7.7] versus 1.5 [95% confidence interval: 0.8-14.2]). Half-lives were calculated using an equation describing a single exponential decay. Statistical analysis using GrapPrism revealed that half-lives were significantly different for WT-FVIII-SQ and FVIII-AT-0203 (p=0.026).

### Example 17: Effect of Multivalent sdAb KB-AT-113 in Thrombin Generation Assay Using Hemophilic Plasma

sdAb KB-AT-443 was analyzed for its capacity to restore thrombin generation in factor VIII (FVIII)-deficient plasma. Thrombin generation was measured according to the method described by Hemker et al (pathophysiology of haemostasis and thrombosis (2002) 32:249-253), in a Fluoroscan Ascent fluorometer (Thermolabsystems OY, Helsink, Finland) equipped with a dispenser. Briefly, 80 microliter of plasma supplemented with either saline (control), purified FVIII (0.1 or 1 U/ml Kogenate® FS, Bayer HealthCare, Puteaux, France) or with KB-AT-443 (4 micromolar) were dispensed into round-bottom 96-well microtiter plates. Twenty microliter of a mixture containing TF (recombinant lipidated human tissue factor, Innovin®, obtained from Dade Behring) and phospholipids (PL) vesicles was added to the plasma sample to obtain a final concentration of 1 pM and 4 μM PL vesicles. PL vesicles were prepared from L-α-Phosphatidyl-L-serine (PS) L-α-phosphatidylethanolamine (PE) and L-α-phosphatidylcholine (PC) (Avanti Polarlipids, Alabaster, Ala., USA) to a ratio of PC:PE:PS=3:1:1 and were of nominal 100 nm-diameter.

Thrombin generation was triggered by adding 20 microliter of starting reagent containing fluorogenic substrate and CaCl2. Fluorogenic substrate I-1140 (Z-Gly-Gly-Arg-AMC) was from Bachem AG (Bubendorf, Switzerland). Kinetics of thrombin generation in clotting plasma was monitored for 60 min at 37° C. using a calibrated automated thrombogram and analyzed using the Thrombinoscope-software (Thrombinoscope B.V., Maastricht, the Netherlands). Four wells were needed for each experiment, two wells to measure thrombin generation of a plasma sample and two wells for calibration. All experiments were carried out in triplicate and the mean value is reported. Endogenous thrombin potential (ETP), i.e. area under the curve, peak thrombin and lag time for thrombin detection were determined.

In FIG. 16, examples of thrombin generation curves are represented. The thrombin-generation parameters are summarized in Table 16. These thrombin generation curves show that KB-AT-443 is similar to FVIII 100% in terms of lag-time and ETP.

| | Lag-time | ETP | Thrombin peak |
|---|---|---|---|
| | (min) | (nM) | (nM) |
| FVIII 0% | 6.3 | 259 | 18 |
| FVIII 10% | 4.3 | 573 | 68 |
| FVIII 100% | 3.3 | 1140 | 166 |
| KB-AT-443 | 3.7 | 1190 | 109 |
| (4 microM) | | | |

### REFERENCES

Throughout this application, various references describe the state of the art to which this invention pertains. The disclosures of these references are hereby incorporated by reference into the present disclosure.

1. An isolated single-domain antibody (sdAb) directed against antithrombin (AT).

2. The isolated single-domain antibody according to claim 1, wherein said sdAb comprises:
a CDR1 having a sequence set forth as SEQ ID NO: 1, a CDR2 having a sequence set forth as SEQ ID NO: 2 and a CDR3 having a sequence set forth as SEQ ID NO: 3;
a CDR1 having a sequence set forth as SEQ ID NO: 5, a CDR2 having a sequence set forth as SEQ ID NO: 6 and a CDR3 having a sequence set forth as SEQ ID NO: 7;
a CDR1 having a sequence set forth as SEQ ID NO: 9, a CDR2 having a sequence set forth as SEQ ID NO: 10 and a CDR3 having a sequence set forth as SEQ ID NO: 11;
a CDR1 having a sequence set forth as SEQ ID NO:13, a CDR2 having a sequence set forth as SEQ ID NO: 14 and a CDR3 having a sequence set forth as SEQ ID NO: 15;
a CDR1 having a sequence set forth as SEQ ID NO: 17, a CDR2 having a sequence set forth as SEQ ID NO: 18 and a CDR3 having a sequence set forth as SEQ ID NO: 19;
a CDR1 having a sequence set forth as SEQ ID NO:21, a CDR2 having a sequence set forth as SEQ ID NO: 22 and a CDR3 having a sequence set forth as SEQ ID NO: 23; or
a CDR1 having a sequence set forth as SEQ ID NO:25, a CDR2 having a sequence set forth as SEQ ID NO: 26 and a CDR3 having a sequence set forth as SEQ ID NO: 27.

3. The isolated single-domain antibody according to claim 1, wherein said sdAb is:
KB-AT-001 (SEQ ID NO: 4),
KB-AT-002 (SEQ ID NO: 8),
KB-AT-003 (SEQ ID NO: 12),
KB-AT-004 (SEQ ID NO: 16),
KB-AT-005 (SEQ ID NO:20),
KB-AT-006 (SEQ ID NO:24) or
KB-AT-007 (SEQ ID NO:28).

4. A drug conjugate comprising the isolated single domain antibody according to claim 1 linked to a heterologous moiety.

5. The drug conjugate according to claim 4 wherein the heterologous moiety is a heterologous polypeptide.

6. The drug conjugate according to claim 5, wherein the heterologous polypeptide is fused to the single domain antibody to form a fusion protein.

7. The drug conjugate according to claim 6, wherein the single domain antibody (sbAb) is fused either directly or via a spacer at its C-terminal end to the N-terminal end of the heterologous polypeptide, or at its N-terminal end to the C-terminal end of the heterologous polypeptide.

8. The drug conjugate according to claim 6, wherein the fusion protein is a biparatopic polypeptide.

9. The drug conjugate according to claim 6, wherein the fusion protein is a biparatopic antibody comprising:
the isolated single domain antibody KB-AT-002 which is linked to the isolated single domain antibody KB-AT-003
the isolated single domain antibody KB-AT-001 which is linked to the isolated single domain antibody KB-AT-002,
the isolated single domain antibody KB-AT-001 which is linked to the isolated single domain antibody KB-AT-003 or
the isolated single domain antibody KB-AT-001 which is linked to the isolated single domain antibody KB-AT-005.

10. The drug conjugate according to claim 6, wherein the fusion protein is a trivalent antibody.

11. The drug conjugate according to claim 10 wherein the trivalent antibody comprises:
two isolated single domain antibodies KB-AT-001 which are linked to the isolated single domain antibody KB-AT-002,
two isolated single domain antibodies KB-AT-001 which are linked to the isolated single domain antibody KB-AT-003, or
two isolated single domain antibodies KB-AT-001 which are linked to the isolated single domain antibody KB-AT-005.

12. The drug conjugate according to claim 6, wherein the fusion protein is a quadrivalent antibody.

13. The drug conjugate according to claim 12 wherein the quadrivalent antibody comprises two isolated single domain antibodies KB-AT-001 which are linked to the isolated single domain antibody KB-AT-002 which is linked to the single domain antibody KB-AT-003.

14. The drug conjugate according to claim 4 wherein the heterologous moiety is a polypeptide.

15. The drug conjugate according to claim 14 wherein the polypeptide is VWF-A1 domain.

16. The drug conjugate according to claim 14 wherein the polypeptide is a polypeptide derived from C4BP.

17. The drug conjugate according to claim 4, wherein the heterologous moiety is a circulating protein.

18. The drug conjugate according to claim 17, wherein the circulating protein is a clotting factor.

19. A vector which comprises the single domain antibody according to claim 1.

20. The vector according to claim 19 wherein the vector is an AAV vector.

21. A method of extending or increasing half-life of a therapeutic polypeptide comprising a step of adding to the polypeptide sequence of said therapeutic polypeptide at least one sdAb directed against antithrombin according to claim 1 which is inserted or not in to a vector.

22. A method of extending or increasing the half-life of the single domain antibody according to claim 1 comprising a step of linking C4BP to the single domain antibody which is inserted or not in to a vector.

23. A method of preventing or treating bleeding disorders in a subject in need thereof, comprising administering to said subject a therapeutically effective amount of the single domain antibody according to claim 1 which is inserted or not in to a vector.

24. The method according to claim 23, wherein the bleeding disorder is hemophilia A or hemophilia B.

25. A method for preventing or treating heparin induced haemorrhages in a subject in need thereof, comprising administering to said subject a therapeutically effective amount of the single domain antibody according to claim 1 which is inserted or not in to a vector.

26. A pharmaceutical composition comprising the single domain antibody according to claim 1, which is inserted or not in to a vector.
